(19) United States
(12) Patent Application Publication
(10) Pub. No.: US 2025/0352822 A1
(43) Pub. Date: Nov. 20, 2025

(54) COMBINATIONS AND USES THEREOF

(71) Applicant: Hoffmann-La Roche Inc., Little Falls, NJ (US)

(72) Inventors: Maria AMANN, Schlieren (CH); Laura CODARRI DEAK, Schlieren (CH); Sana Dole KARAM, Aurora, CO (US); Christian KLEIN, Schlieren (CH); Pablo UMAÑA FERNÁNDEZ, Schlieren (CH)

(21) Appl. No.: 19/289,210

(22) Filed: Aug. 4, 2025

Related U.S. Application Data
(63) Continuation of application No. PCT/EP2024/052679, filed on Feb. 5, 2024.
(60) Provisional application No. 63/443,466, filed on Feb. 6, 2023.

(51) Int. Cl. A61N 5/10; A61P 35/04; C07K 16/28

(52) U.S. Cl. CPC A61N 5/1048; A61P 35/04; C07K 16/2896; C07K 2317/31

(57) ABSTRACT

The present invention provides novel combination therapies comprising radio therapy, a bispecific immunocytokine and, optionally, a Treg depleting agent. The invention also provides methods of using said combination therapy in the treatment of cancer, preferably solid tumors.

### CROSS REFERENCE TO RELATED APPLICATIONS

This application is a Continuation of PCT Application No. PCT/EP2024/052679 filed Feb. 5, 2024, which claims priority from U.S. Provisional Applications 63/443,466 filed on Feb. 6, 2023. The priority of said PCT and US Provisional Application are claimed. Each of the prior mentioned applications is hereby incorporated by reference herein in its entirety.

### REFERENCE TO AN ELECTRONIC SEQUENCE LISTING

The instant application contains a Sequence Listing which has been submitted electronically in XML file format and is hereby incorporated by reference in its entirety. Said XML copy, created on Aug. 1, 2025, is named P38227_WO_Sequence listing.xml and is 18,503 bytes in size.

### FIELD OF THE INVENTION

The present invention relates to combination therapies of PD-1-targeted IL-2 variant immunoconjugates, radiotherapy and, optionally, depletion of regulatory T-cells (Treg's), preferably by using non-Il-2 blocking anti-CD25 antibodies. The present invention also relates to the use of these combination therapies for the treatment of solid tumors.

### BACKGROUND OF THE INVENTION

The advent of cancer immunotherapy has revolutionized the way oncologists manage and treat cancer, increasing response rates and overall survival of patients. However, the success of immunotherapies, most notably immune checkpoint inhibitors, is not all encompassing, with varying levels of efficacy between cancer types or even among patients with the same malignancy¹. Head and neck squamous cell carcinomas (HNSCC) are among the most prevalent malignancies worldwide² and can be characterized by their immunologically cold tumors and high resistance to therapy, ultimately resulting in poor treatment outcomes³. Radiation therapy is used as standard of care in many cases and, in addition to direct tumor cell kill, helps sensitize the tumor microenvironment (TME) to immunotherapy⁴. Despite these efforts, the development of acquired resistance to combination radioimmunotherapy invariably occurs, with full understanding of the underlying mechanisms yet to be determined.

The immune system plays a fundamental role in disease progression and treatment response in HNSCC^{4,5}. Radiation therapy (RT) induces an influx of proinflammatory immune cells into the TME⁴. However, this induction of a proinflammatory TME is transient, ultimately being dampened by the influx of immunosuppressive regulatory T cells (Tregs)^{4,6}. Tregs have been identified as key regulators of resistance to radioimmunotherapy⁷⁻⁹, and can indirectly suppress anti-tumor immunity through the sequestration of IL-2. Ablation of Tregs using αCD25 can result in the activation of natural killer cells (NK), a process that is mediated by IL-2 signaling through CD122^{10,11}. NK cells, a class of innate lymphoid cells, play a prominent role in tumor surveillance and direct cytotoxic cell kill both locally and in the periphery^{12,13}. Furthermore, NK cells are reliant upon IL-2 signaling for modulating much of their homeostatic and cytotoxic potential^{14,15}, making them an enticing target for IL-2 directed immunotherapy.

Immune exhaustion is another confounding factor of acquired resistance¹⁶. High intratumoral surface expression of programmed cell death protein 1 (PD-1) on lymphocytes coupled with the upregulation of programmed cell death ligand 1 (PD-L1) on tumor cells and suppressive immune cells, ensures that tumor infiltrating lymphocytes are incapable of achieving a sustained anti-tumor response^{4,6,17}. While PD-1 blockade has been shown to restore PD-1⁺ T effector functionality, it also possesses the dichotomous effect of amplifying Treg mediated immunosuppression among PD-1⁺ Tregs^{18,19}. Therefore, the reactivation of PD-1⁺ effector T cells with αPD-1 therapy may only be achievable through the subsequent depletion of Tregs.

Pancreatic ductal adenocarcinoma (PDAC) is a malignancy known to establish an immunosuppressive tumor microenvironment (TME) making it resistant to conventional targeted and cytotoxic therapies, such as radiation therapy (RT) (Dougan (2017); (Quiñonero et al., 2019). Even in the face of immune-invigorating treatments, responses in this disease type are almost always transient (Molejon et al., 2015). Examination of the biological elements contributing to immune escape in the setting of treatment failure, therefore, is imperative to overcoming inherent resistance. Given that PDAC is a systemic disease with a high risk of metastasis, and with only approximately 10% of patients diagnosed at early stages (Zhu et al., 2018), any treatment aimed at improving response and outcomes must place emphasis on systemic immunity. With the practice of immunotherapy in PDAC still in its infancy there remains a need for improved treatment of this deadly disease.

### FIGURES

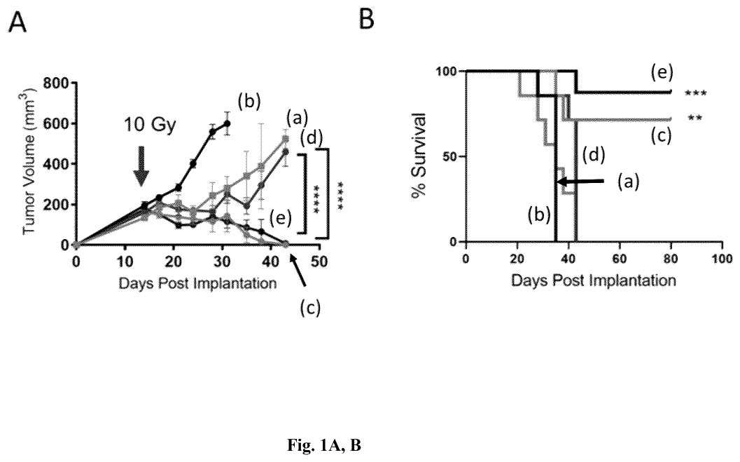

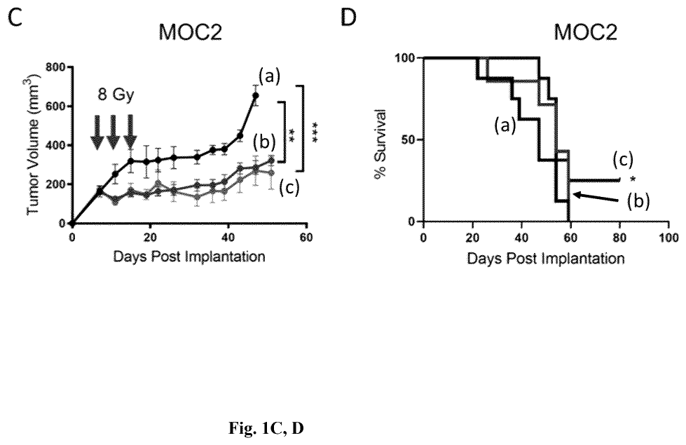

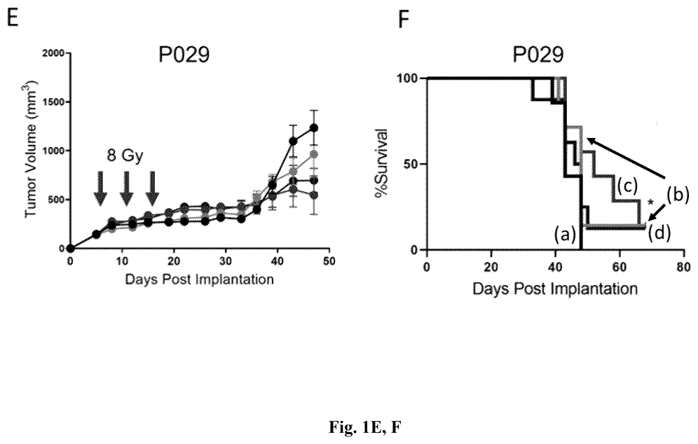

FIG. 1: Treatment with αCD25 and PD1-IL2v results in tumor growth delay in HNSCC tumors
A) Tumor growth curves for mice implanted with LY2 tumors.
B) Kaplan Meier survival curve of mice implanted with LY2 tumors. RT:RT+αCD25: Hazard Ratio=4.943; 95% CI=1.312-18.62. RT:RT+αCD25+PD1-IL2v: Hazard Ratio=11.43; 95% CI=2.742-47.67.
LY2 tumors were established and treated with (a) PD1-IL2v ( 0/7), (b) RT ( 0/7), (c) RT+αCD25 ( 5/7), (d) RT+PD1-IL2v ( 0/7), (e) RT+αCD25+PD1-IL2v (⅞). RT was delivered as a single dose of 10 Gy. Antibodies were administered weekly via i.p. injection for the duration of the study.
C) Tumor growth curves for mice implanted with MOC2 tumors.
D) Kaplan Meier survival curve of mice implanted with MOC2 tumors. RT:RT+αCD25+PD1-IL2v: Hazard Ratio=2.388; 95% CI=0.801-7.121.
MOC2 tumors were established and treated with (a) RT ( 0/8), (b) RT+PD1-IL2v ( 0/8), (c) RT+αCD25+PD1-IL2v ( 2/8). RT was delivered in 3 fractions of 8 Gy, with 3-4 days between fractions. Antibodies were administered weekly via i.p. injection for the duration of the study.
E) Tumor growth curves for mice implanted with P029 tumors
F) Kaplan Meier survival curve of mice implanted with P029 tumors. RT:RT+αCD25+PD1-IL2v: Hazard Ratio=2.354; 95% CI=0.7418-7.471.
P029 tumors were established and treated with (a) RT ( 0/7), (b) RT+αCD25 ( 1/7), (c) RT+PD1-IL2v ( 1/7), (d) RT+αCD25+PD1-IL2v (⅛). RT was delivered in 3 fractions of 8 Gy, with 5 days between fractions. Antibodies were administered weekly via i.p. injection for the duration of the study.
For all FIG. 1: n=7-8 for all in vivo experiments. All statistical analysis was performed using one-way ANOVA unless stated otherwise. * P<0.05, ** P<0.005, *** P<0.0005, **** P<0.0001

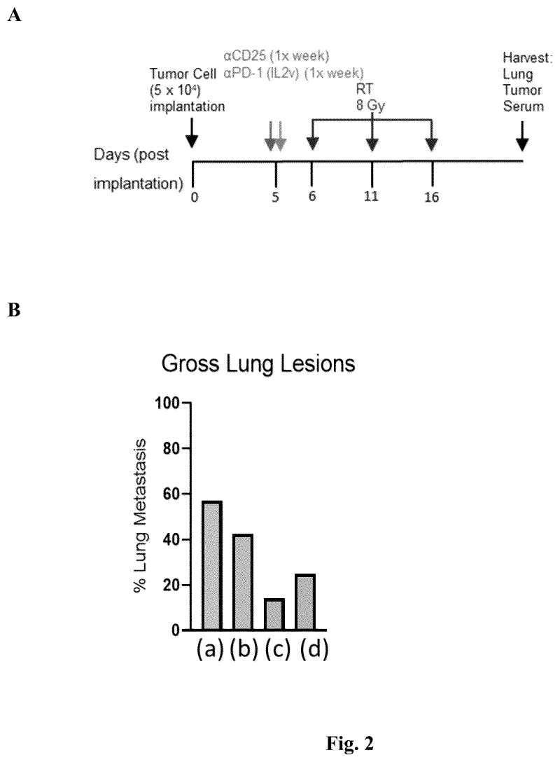

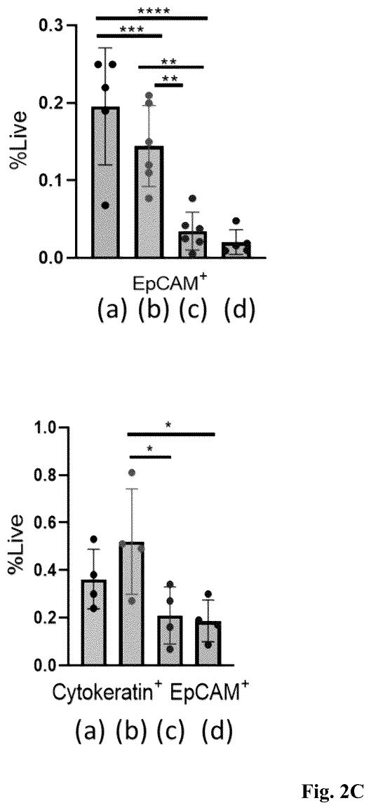

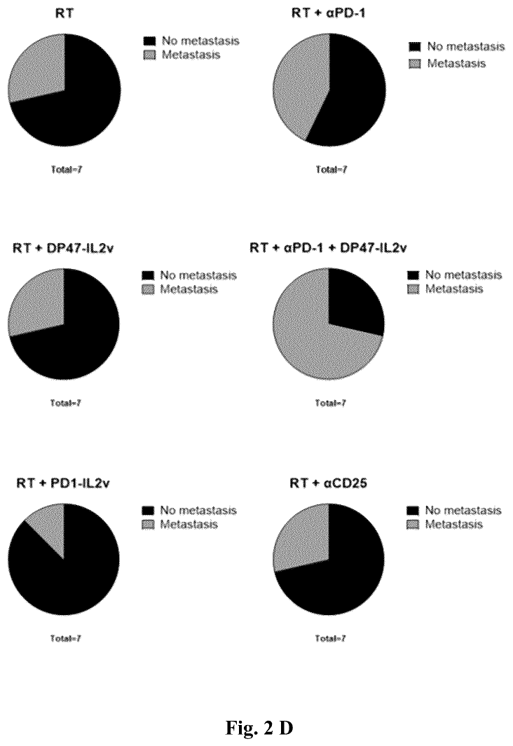

FIG. 2: Radiation Therapy and PD1-IL2v abrogates lung metastasis
A) Schematic describing experimental design. P029 tumors were established in C57 B1/6 mice and treated with RT and a combination of αCD25, PD1-IL2v, αCD25 and PD1-IL2v, or no immunotherapy. RT was delivered as 3 fractions of 8 Gy RT, 5 days apart. Antibodies were administered weekly via i.p. injection beginning one day prior to RT.
B) Percentage of mice from each treatment group exhibiting gross lung metastases. n=7
C) Frequency of circulating tumor cells (CTC's) in the blood of tumor bearing P029 mice. CTC's are defined as CD45-EpCAM+ and CD45-EpCAM+ pan-Cytokeratin+
D) The proportion of mice implanted with P029 tumors which exhibit metastasis to the lungs among treatment groups.
All statistical analysis was performed using one-way ANOVA unless stated otherwise. * P<0.05, ** P<0.005, *** P<0.0005, **** P<0.0001

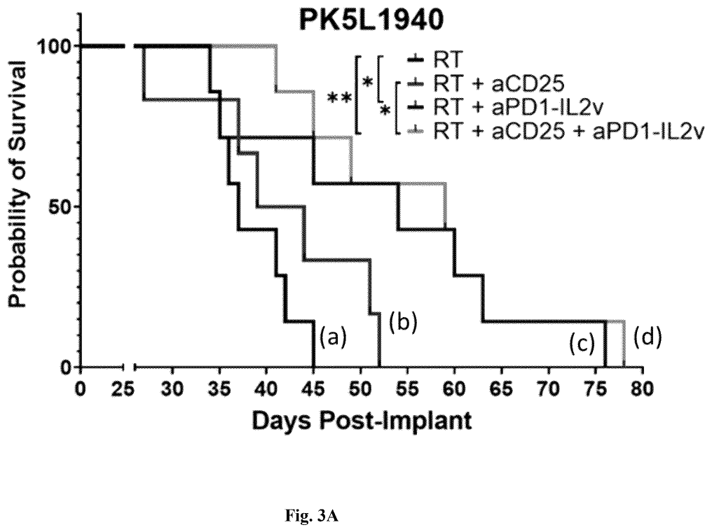

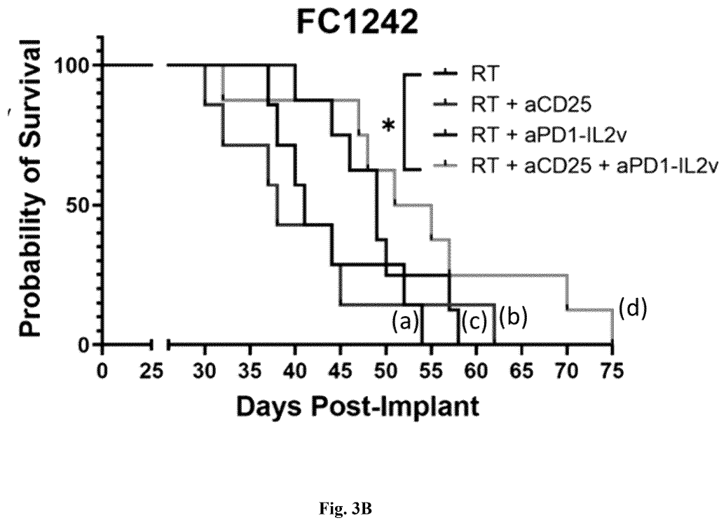

FIG. 3: Survival of pre-clinical murine PDAC models
A) Kaplan-Meier survival analysis of PK5L1940 orthotopically implanted pancreatic tumor-bearing mice treated with (a) RT, (b) RT+aCD25, (c) RT+aPD1-IL2v, (d) RT+aCD25+aPD1-IL2v.
B) Kaplan-Meier survival analysis of FC1242 orthotopically implanted pancreatic tumor-bearing mice treated with (a) RT, (b) RT+aCD25, (c) RT+aPD1-IL2v, (d) RT+aCD25 +aPD1-IL2v.
RT was administered 7 days post-implantation. aPD1-IL2v and aCD25 dosed once per week beginning day 7 post-implantation. n≥7 per group. P-values calculated with Students T-test, *indicates p<0.05, **<0.01.

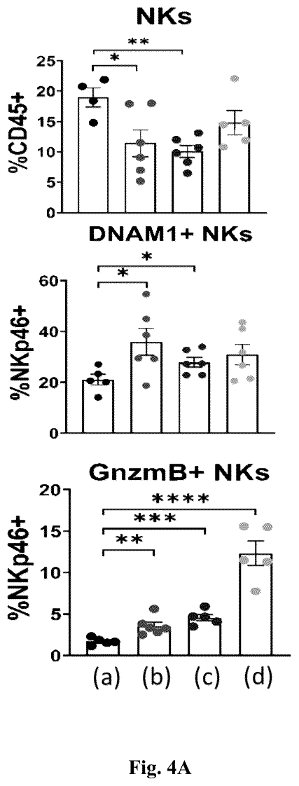

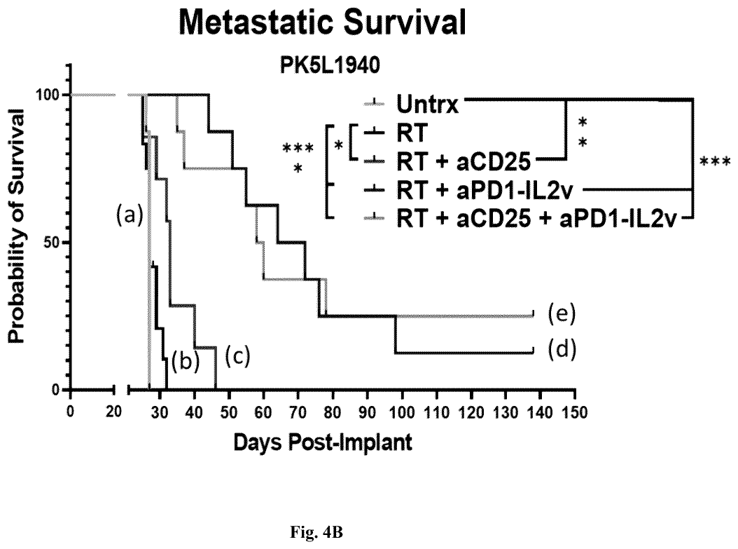

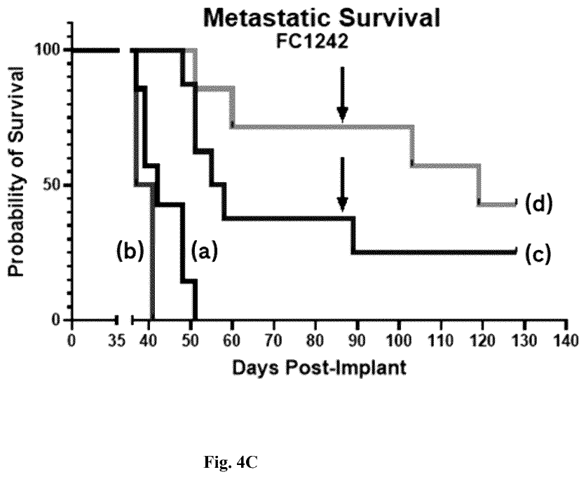

FIG. 4: Peripheral activation of anti-tumor immune populations reduces metastatic burden in PDAC models
A) Flow cytometric analysis of the frequency of NK cells (top), and NK cell expression of functional markers DNAM1 (center) and Gnzmb (bottom) in the blood of pancreatic tumor-bearing mice treated with (a) RT, (b) RT+aCD25, (c) RT+aPD1-IL2v and (d) RT+aCD25+aPD1-IL2v. n≥5 per group. P-values calculated with Students T-test, *indicates p<0.05, **<0.01, ***<0.001, ****<0.0001.
B) Kaplan-Meier survival analysis of mice implanted with PK5L1940 using a hemi-splenectomy model of metastatic pancreatic cancer. Mice were (a) untreated or treated with (b) RT, (c) RT+aCD25, (d) RT+aPD1-IL2v, (e) RT+aCD25 +aPD1-IL2v. n≥7 per group. P-values were calculated with Students T-test, *indicates p<0.05, **<0.01, ***<0.001, ****<0.0001.
C) Kaplan-Meier survival analysis of mice implanted with FC1242 cells using a hemi-splenectomy model of metastatic pancreatic cancer. RT administered 7 days post-implantation. PD1-IL2v and aCD25 dosed once per week beginning day 7 post-implantation Treatment groups: (a) RT alone (n≥7), (b) RT+aCD25 (n<7), (c) RT+PD1-IL2v (n≥7), and (d) RT+aCD25 +PD1-IL2v (n≥7). P-values calculated with Students T-test, (a) to (c) <0.01, (d) <0.001.

### DETAILED DESCRIPTION OF THE INVENTION

The present invention provides a combination for use in the treatment of cancer, wherein the combination comprises a) a first component comprising an effective amount of a Treg cell depleting agent; and b) a second component comprising an effective amount of a bispecific immunocytokine; and c) a third component comprising an effective amount of radiation therapy, and wherein the components a) to c) are for simultaneous or sequential administration.

In one embodiment, the use of the first component a) is optional. Thus in another embodiment, the present invention provides a combination for use in the treatment of cancer, wherein the combination comprises an effective amount of a bispecific immunocytokine and an effective amount of radiation therapy for simultaneous or sequential administration.

Unless defined otherwise, technical and scientific terms used herein have the same meaning as generally used in the art to which this invention belongs. For purposes of interpreting this specification, the following definitions will apply and whenever appropriate, terms used in the singular will also include the plural and vice versa.

### Bispecific Immunocytokines

The bispecific immunocytokines used in the combination therapy described herein comprise a PD-1-targeted antigen binding moiety and an IL-2-based effector moiety, for example including a mutant IL-2, are described in e.g. WO 2018/184964. In one aspect, the PD-1-targeted antigen binding moiety comprises an antibody which binds to PD-1 on PD-1 expressing immune cells, particularly T cells, or in a tumor cell environment, or an antigen binding fragment thereof, and an IL-2 mutant, particularly a mutant of human IL-2, having reduced binding affinity to the α-subunit of the IL-2 receptor (as compared to wild-type IL-2, e.g. human IL-2 shown as SEQ ID NO: 4), such as an IL-2 comprising: i) one, two or three amino acid substitution(s) at one, two or three position(s) selected from the positions corresponding to residues 42, 45 and 72 of human IL-2 shown as SEQ ID NO: 4, for example three substitutions at three positions, for example the specific amino acid substitutions F42A, Y45A and L72G; or ii) the features as set out in i) plus an amino acid substitution at a position corresponding to residue 3 of human IL-2 shown as SEQ ID NO: 4, for example the specific amino acid substitution T3A; or iii) four amino acid substitutions at positions corresponding to residues 3, 42, 45 and 72 of human IL-2 shown as SEQ ID NO: 4, for example the specific amino acid substitutions T3A, F42A, Y45A and L72G. In one embodiment, the antibody may be an IgG antibody, particularly an IgG1 antibody. In another embodiment, the PD-1-targeted IL-2 variant immunoconjugate may comprise a single IL-2 mutant having reduced binding affinity to the subunit of the IL-2 receptor (i.e. not more than one IL-2 mutant moiety is present).

In preferred embodiments, PD-1 targeting of the PD-1-targeted IL-2 variant immunoconjugate may be achieved by targeting PD-1, as described in WO 2018/1184964. PD-1-targeting may be achieved with an anti-PD-1 antibody or an antigen binding fragment thereof. An anti-PD-1 antibody may comprise a heavy chain variable region sequence that is at least about 80%, 85%, 90%, 95%, 96%, 97%, 98%, 99% or 100% identical to the sequence of SEQ ID NO: 1 or a variant thereof that retains functionality. An anti-PD-1 antibody may comprise a light chain variable region sequence that is at least about 80%, 85%, 90%, 95%, 96%, 97%, 98%, 99% or 100% identical to the sequence of SEQ ID NO: 2 or a variant thereof that retains functionality. An anti-PD-1 antibody may comprise a heavy chain variable region sequence that is at least about 80%, 85%, 90%, 95%, 96%, 97%, 98%, 99% or 100% identical to the sequence of SEQ ID NO: 1, or a variant thereof that retains functionality, and a light chain variable region sequence that is at least about 80%, 85%, 90%, 95%, 96%, 97%, 98%, 99% or 100% identical to the sequence of SEQ ID NO: 2, or a variant thereof that retains functionality. An anti-PD-1 antibody may comprise the heavy chain variable region sequence of SEQ ID NO: 1 and the light chain variable region sequence of SEQ ID NO: 2.

The PD-1-targeted IL-2 variant immunoconjugate may comprise a polypeptide sequence selected from the group consisting of SEQ ID NO: 5, SEQ ID NO: 6 and SEQ ID NO: 7, or a variant thereof that retains functionality. The PD-1-targeted IL-2 variant immunoconjugate may comprise a polypeptide sequence wherein a Fab heavy chain specific for PD-1 shares a carboxy-terminal peptide bond with an Fc domain subunit comprising a hole modification. The PD-1-targeted IL-2 variant immunoconjugate may comprise the polypeptide sequence of SEQ ID NO: 5 or SEQ ID NO: 6, or a variant thereof that retains functionality. The PD-1-targeted IL-2 variant immunoconjugate may comprise a Fab light chain specific for PD-1. The PD-1-targeted IL-2 variant immunoconjugate may comprise the polypeptide sequence of SEQ ID NO: 7, or a variant thereof that retains functionality. The polypeptides may be covalently linked, e.g., by a disulfide bond. The Fc domain polypeptide chains may comprise the amino acid substitutions L234A, L235A, and P329G (which may be referred to as LALA P329G).

As described in WO 2018/184964, the PD-1-targeted IL-2 variant immunoconjugate may be a PD-1-targeted IgG-IL-2 qm fusion protein having the sequences shown as SEQ ID NOs: 5, 6, 7 (as described in e.g. Example 1 of WO 2018/184964). The PD-1-targeted IL-2 variant immunoconjugate having the sequences shown as SEQ ID NOs: 5, 6, 7 is referred to herein as “PD1-IL2v”. The PD-1-targeted IL-2 variant immunoconjugate having the sequences shown as SEQ ID NOs: 8, 9, 10 is referred to herein as “muPD1-IL2v”, which is a murine surrogate.

In another embodiment, a PD-1-targeted IL-2 variant immunoconjugate used in the present combination therapy may comprise a) a heavy chain variable domain VH of SEQ ID NO: 1 and a light chain variable domain VL of SEQ ID NO: 2, and the polypeptide sequence of SEQ ID NO: 3, or b) the polypeptide sequences of SEQ ID NO: 5, and SEQ ID NO: 6 and SEQ ID NO: 7, or c) the polypeptide sequences of SEQ ID NO: 8, and SEQ ID NO: 9 and SEQ ID NO: 10.

As described herein, the PD-1-targeted IL-2 variant immunoconjugate and antigen binding molecules used in the combination therapy described herein may comprise an Fc domain consisting of two subunits and comprising a modification promoting heterodimerization of two non-identical polypeptide chains. The PD-1-targeted IL-2 variant immunoconjugate and the antigen binding molecules used in the combination therapy described herein may comprise an Fc domain subunit comprising a knob mutation and an Fc domain subunit comprising a hole mutation.

A “modification promoting heterodimerization” is a manipulation of the peptide backbone or the post-translational modifications of a polypeptide that reduces or prevents the association of the polypeptide with an identical polypeptide to form a homodimer. A modification promoting heterodimerization as used herein particularly includes separate modifications made to each of two polypeptides desired to form a dimer, wherein the modifications are complementary to each other so as to promote association of the two polypeptides. For example, a modification promoting heterodimerization may alter the structure or charge of one or both of the polypeptides desired to form a dimer so as to make their association sterically or electrostatically favorable, respectively. Heterodimerization occurs between two non-identical polypeptides, such as two subunits of an Fc domain wherein further immunoconjugate components fused to each of the subunits (e.g. antigen binding moiety, effector moiety) are not the same. In the immunoconjugates and bispecific antibodies according to the present invention, the modification promoting heterodimerization is in the Fc domain. In some embodiments the modification promoting heterodimerziation comprises an amino acid mutation, specifically an amino acid substitution. In a particular embodiment, the modification promoting heterodimerization comprises a separate amino acid mutation, specifically an amino acid substitution, in each of the two subunits of the Fc domain. The site of most extensive protein-protein interaction between the two polypeptide chains of a human IgG Fc domain is in the CH3 domain of the Fc domain. Thus, in one embodiment said modification is in the CH3 domain of the Fc domain. In a specific embodiment said modification is a knob-into-hole modification, comprising a knob modification in one of the two subunits of the Fc domain and a hole modification in the other one of the two subunits of the Fc domain.

The knob-into-hole technology is described e.g. in U.S. Pat. No. 5,731,168; U.S. Pat. No. 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine). The protuberance and cavity can be made by altering the nucleic acid encoding the polypeptides, e.g. by site-specific mutagenesis, or by peptide synthesis. In a specific embodiment a knob modification comprises the amino acid substitution T366W in one of the two subunits of the Fc domain, and the hole modification comprises the amino acid substitutions T366S, L368A and Y407V in the other one of the two subunits of the Fc domain. In a further specific embodiment, the subunit of the Fc domain comprising the knob modification additionally comprises the amino acid substitution S354C, and the subunit of the Fc domain comprising the hole modification additionally comprises the amino acid substitution Y349C. Introduction of these two cysteine residues results in formation of a disulfide bridge between the two subunits of the Fc region, further stabilizing the dimer (Carter, J Immunol Methods 248, 7-15 (2001)). Numbering of amino acid residues in the Fc region is according to the EU numbering system, also called the EU index, as described in Kabat et al., Sequences of Proteins of Immunological Interest, 5th Ed. Public Health Service, National Institutes of Health, Bethesda, MD, 1991. A “subunit” of an Fc domain as used herein refers to one of the two polypeptides forming the dimeric Fc domain, i.e. a polypeptide comprising C-terminal constant regions of an immunoglobulin heavy chain, capable of stable self-association. For example, a subunit of an IgG Fc domain comprises an IgG CH2 and an IgG CH3 constant domain.

In an alternative embodiment a modification promoting heterodimerization of two non-identical polypeptide chains comprises a modification mediating electrostatic steering effects, e.g. as described in WO 2009/089004. Generally, this method involves replacement of one or more amino acid residues at the interface of the two polypeptide chains by charged amino acid residues so that homodimer formation becomes electrostatically unfavorable but heterodimerization electrostatically favorable.

An IL-2 mutant having reduced binding affinity to the subunit of the IL-2 receptor may be fused to the carboxy-terminal amino acid of the subunit of the Fc domain comprising the knob modification. Without wishing to be bound by theory, fusion of the IL-2 mutant to the knob-containing subunit of the Fc domain will further minimize the generation of homodimeric immunoconjugates comprising two IL-2 mutant polypeptides (steric clash of two knob-containing polypeptides).

The Fc domain of the immunoconjugate and antigen binding molecules may be engineered to have altered binding affinity to an Fc receptor, specifically altered binding affinity to an Fcγ receptor, as compared to a non-engineered Fc domain, as described in WO 2012/146628. Binding of the Fc domain to a complement component, specifically to C1q, may be altered, as described in WO 2012/146628. The Fc domain confers to the immunoconjugate and bispecific antibodies favorable pharmacokinetic properties, including a long serum half-life which contributes to good accumulation in the target tissue and a favorable tissue-blood distribution ratio. At the same time it may, however, lead to undesirable targeting to cells expressing Fc receptors rather than to the preferred antigen-bearing cells. Moreover, the co-activation of Fc receptor signaling pathways may lead to cytokine release which, in combination with the effector moiety and the long half-life of the immunoconjugate, results in excessive activation of cytokine receptors and severe side effects upon systemic administration. In line with this, conventional IgG-IL-2 immunoconjugates have been described to be associated with infusion reactions (see e.g. King et al., J Clin Oncol 22, 4463-4473 (2004)).

Accordingly, the Fc domain of the immunoconjugate and antigen binding molecules may be engineered to have reduced binding affinity to an Fc receptor. In one such embodiment the Fc domain comprises one or more amino acid mutation that reduces the binding affinity of the Fc domain to an Fc receptor. Typically, the same one or more amino acid mutation is present in each of the two subunits of the Fc domain. In one embodiment said amino acid mutation reduces the binding affinity of the Fc domain to the Fc receptor by at least 2-fold, at least 5-fold, or at least 10-fold. In embodiments where there is more than one amino acid mutation that reduces the binding affinity of the Fc domain to the Fc receptor, the combination of these amino acid mutations may reduce the binding affinity of the Fc domain to the Fc receptor by at least 10-fold, at least 20-fold, or even at least 50-fold. In one embodiment the immunoconjugate and bispecific antibodies comprising an engineered Fc domain exhibit less than 20%, particularly less than 10%, more particularly less than 5% of the binding affinity to an Fc receptor as compared to immunoconjugates and bispecific antibodies comprising a non-engineered Fc domain. In one embodiment the Fc receptor is an activating Fc receptor. In a specific embodiment the Fc receptor is an Fcγ receptor, more specifically an Fcγ RIIIa, Fcγ RI or Fcγ RIIa receptor. Preferably, binding to each of these receptors is reduced. In some embodiments binding affinity to a complement component, specifically binding affinity to C1q, is also reduced. In one embodiment binding affinity to neonatal Fc receptor (FcRn) is not reduced. Substantially similar binding to FcRn, i.e. preservation of the binding affinity of the Fc domain to said receptor, is achieved when the Fc domain (or the immunoconjugate comprising said Fc domain) exhibits greater than about 70% of the binding affinity of a non-engineered form of the Fc domain (or the immunoconjugate comprising said non-engineered form of the Fc domain) to FcRn. Fc domains, or immunoconjugates and bispecific antibodies of the invention comprising said Fc domains, may exhibit greater than about 80% and even greater than about 90% of such affinity. In one embodiment the amino acid mutation is an amino acid substitution. In one embodiment the Fc domain comprises an amino acid substitution at position P329. In a more specific embodiment the amino acid substitution is P329A or P329G, particularly P329G. In one embodiment the Fc domain comprises a further amino acid substitution at a position selected from S228, E233, L234, L235, N297 and P331. In a more specific embodiment the further amino acid substitution is S228P, E233P, L234A, L235A, L235E, N297A, N297D or P331S. In a particular embodiment the Fc domain comprises amino acid substitutions at positions P329, L234 and L235. In a more particular embodiment the Fc domain comprises the amino acid mutations L234A, L235A and P329G (LALA P329G). This combination of amino acid substitutions almost completely abolishes Fcγ receptor binding of a human IgG Fc domain, as described in WO 2012/130831, incorporated herein by reference in its entirety. WO 2012/130831 also describes methods of preparing such mutant Fc domains and methods for determining its properties such as Fc receptor binding or effector functions. Numbering of amino acid residues in the Fc region is according to the EU numbering system, also called the EU index, as described in Kabat et al., Sequences of Proteins of Immunological Interest, 5th Ed. Public Health Service, National Institutes of Health, Bethesda, MD, 1991.

Mutant Fc domains can be prepared by amino acid deletion, substitution, insertion or modification using genetic or chemical methods well known in the art and as described in WO 2012/146628. Genetic methods may include site-specific mutagenesis of the encoding DNA sequence, PCR, gene synthesis, and the like. The correct nucleotide changes can be verified for example by sequencing.

In one embodiment the Fc domain is engineered to have decreased effector function, compared to a non-engineered Fc domain, as described in WO 2012/146628. The decreased effector function can include, but is not limited to, one or more of the following: decreased complement dependent cytotoxicity (CDC), decreased antibody-dependent cell-mediated cytotoxicity (ADCC), decreased antibody-dependent cellular phagocytosis (ADCP), decreased cytokine secretion, decreased immune complex-mediated antigen uptake by antigen-presenting cells, decreased binding to NK cells, decreased binding to macrophages, decreased binding to monocytes, decreased binding to polymorphonuclear cells, decreased direct signaling inducing apoptosis, decreased crosslinking of target-bound antibodies, decreased dendritic cell maturation, or decreased T cell priming.

IgG4 antibodies exhibit reduced binding affinity to Fc receptors and reduced effector functions as compared to IgG1 antibodies. Hence, in some embodiments the Fc domain of the antigen binding molecules of the invention are an IgG4 Fc domain, particularly a human IgG4 Fc domain. In one embodiment the IgG4 Fc domain comprises amino acid substitutions at position S228, specifically the amino acid substitution S228P. To further reduce its binding affinity to an Fc receptor and/or its effector function, in one embodiment the IgG4 Fc domain comprises an amino acid substitution at position L235, specifically the amino acid substitution L235E. In another embodiment, the IgG4 Fc domain comprises an amino acid substitution at position P329, specifically the amino acid substitution P329G. In a particular embodiment, the IgG4 Fc domain comprises amino acid substitutions at positions S228, L235 and P329, specifically amino acid substitutions S228P, L235E and P329G. Such IgG4 Fc domain mutants and their Fcγ receptor binding properties are described in European patent application no. WO 2012/130831, incorporated herein by reference in its entirety.

### Radiotherapy

In aspects of the present invention, the PD1-IL2v immunoconjugate described herein is used in combination with radiotherapy.

Radiotherapy or radiation therapy (“RT”) is a therapy using ionizing radiation, generally as part of cancer treatment to control or kill malignant cells and normally delivered by a linear accelerator. Radiotherapy may be curative in a number of types of cancer if they are localized to one area of the body. It may also be used as part of adjuvant therapy, to prevent tumor recurrence after surgery to remove a primary malignant tumor (for example, early stages of breast cancer). Radiation therapy is synergistic with chemotherapy, and has been used before, during, and after chemotherapy in susceptible cancers.

Radiotherapy is commonly applied to the cancerous tumor because of its ability to control cell growth. Ionizing radiation works by damaging the DNA of cancerous tissue leading to cellular death. To spare normal tissues (such as skin or organs which radiation must pass through to treat the tumor), shaped radiation beams are aimed from several angles of exposure to intersect at the tumor, providing a much larger absorbed dose there than in the surrounding healthy tissue. Besides the tumor itself, the radiation fields may also include the draining lymph nodes if they are clinically or radiologically involved with the tumor, or if there is thought to be a risk of subclinical malignant spread. It is necessary to include a margin of normal tissue around the tumor to allow for uncertainties in daily set-up and internal tumor motion. These uncertainties can be caused by internal movement and movement of external skin marks relative to the tumor position. The response of a cancer to radiation is described by its radiosensitivity. Highly radiosensitive cancer cells are rapidly killed by modest doses of radiation. These include leukemias, most lymphomas and germ cell tumors. The majority epithelial cancers are only moderately radiosensitive, and require a significantly higher dose of radiation (60-70 Gy) to achieve a radical cure. Some types of cancer are notably radioresistant, that is, much higher doses are required to produce a radical cure than may be safe in clinical practice. It is important to distinguish the radiosensitivity of a particular tumor, which to some extent is a laboratory measure, from the radiation “curability” of a cancer in actual clinical practice. For example, leukemias are not generally curable with radiation therapy, because they are disseminated through the body. Lymphoma may be radically curable if it is localised to one area of the body. Similarly, many of the common, moderately radioresponsive tumors are routinely treated with curative doses of radiation therapy if they are at an early stage. Cancers such as skin cancer, head and neck cancer, breast cancer, non-small cell lung cancer, cervical cancer and prostate cancer are often incurable with radiotherapy because it is not possible to treat the whole body.

The response of a tumor to radiotherapy depends on the size of the tumor. Very large tumors respond less well to radiation than smaller tumors or microscopic disease. Various strategies are used to overcome this effect. The most common technique is surgical resection prior to radiation therapy. This is most commonly seen in the treatment of breast cancer with mastectomy followed by adjuvant radiotherapy. Another method is to shrink the tumor with neoadjuvant chemotherapy prior to radical radiation therapy. A third technique is to enhance the radiosensitivity of the cancer by giving certain drugs during a course of radiation therapy. Examples of radiosensitizing drugs include cisplatin, nimorazole and cetuximab.

Radiotherapy usually causes minimal or no side effects, although short-term pain flare-up can be experienced in the days following treatment due to oedema compressing nerves in the treated area. Higher doses can cause varying side effects during treatment (acute side effects), in the months or years following treatment (long-term side effects), or after re-treatment (cumulative side effects). The nature, severity, and longevity of side effects depends on the organs that receive the radiation, the treatment itself (type of radiation, dose, fractionation), and the patient. The main side effects reported are fatigue and skin irritation, like a mild to moderate sun burn. The fatigue often sets in during the middle of a course of treatment and can last for weeks after treatment ends. The irritated skin will heal, but may not be as elastic as it was before. Side effects from radiation are often limited to the area of the patient's body that is under treatment and are dose- dependent. For example, higher doses of head and neck radiation can be associated with cardiovascular complications, thyroid dysfunction, and pituitary axis dysfunction. Thus, a lower dose of radiotherapy may be preferred.

The amount of radiation used in photon radiotherapy is measured in Grays (Gy), and varies depending on the type and stage of cancer being treated. For curative cases, the typical dose for a solid epithelial tumor ranges from 60 to 80 Gy, while lymphomas are treated with a range of 20 to 40 Gy. Preventive doses are typically in the range of 45 to 60 Gy in fractions of 1.8 to 2 Gy (for breast, head, and neck cancers.) Many other factors are considered by radiation oncologists when selecting a dose, including whether the patient is receiving chemotherapy, patient comorbidities, whether radiotherapy is being administered before or after surgery. Delivery parameters of a prescribed dose are determined during treatment planning (part of dosimetry). Treatment planning is generally performed on dedicated computers using specialized treatment planning software. Depending on the radiation delivery method, several angles or sources may be used to sum to the total necessary dose. The planner will try to design a plan that delivers a uniform prescription dose to the tumor and minimizes the dose to surrounding healthy tissues.

The total dose is fractionated (spread out over time) for several important reasons. Fractionation allows normal cells time to recover, while tumor cells are generally less efficient in repair between fractions. Fractionation also allows tumor cells that were in a relatively radio-resistant phase of the cell cycle during one treatment to cycle into a sensitive phase of the cycle before the next fraction is given. Similarly, tumor cells that were chronically or acutely hypoxic (and therefore more radioresistant) may reoxygenate between fractions, improving the tumor cell kill. The typical fractionation schedule for adults is 1.8 to 2 Gy per day, five days a week. In some cancer types, prolongation of the fraction schedule over too long can allow for the tumor to begin repopulating, and for these tumor types, including head-and-neck and cervical squamous cell cancers, radiation treatment is preferably completed within a certain amount of time. For children, a typical fraction size may be 1.5 to 1.8 Gy per day, as smaller fraction sizes are associated with reduced incidence and severity of late-onset side effects in normal tissues. In some cases, two fractions per day are used near the end of a course of treatment. This schedule, known as a concomitant boost regimen or hyperfractionation, is used on tumors that regenerate more quickly when they are smaller. In particular, tumors in the head-and-neck demonstrate this behavior.

One fractionation schedule that is increasingly being used and continues to be studied is hypofractionation. This is a radiation treatment in which the total dose of radiation is divided into large doses. Typical doses vary significantly by cancer type, from 1.8 Gy/fraction to 20 Gy/fraction, the latter being typical of stereotactic treatments (stereotactic ablative body radiotherapy, or SABR—also known as SBRT, or stereotactic body radiotherapy) for subcranial lesions, or SRS (stereotactic radiosurgery) for intracranial lesions. A hypofractioned radiation at a dose in a range of 5 to 20 Gy may be preferred. Depending on the cancer to be treated, a hypofractioned radiation at a dose in a range of 1.8 to 2.2 Gy may be of particular interest. The rationale of hypofractionation is to reduce the probability of local recurrence by denying clonogenic cells the time they require to reproduce and also to exploit the radiosensitivity of some tumors. In particular, stereotactic treatments are intended to destroy clonogenic cells by a process of ablation—i.e. the delivery of a dose intended to destroy clonogenic cells directly, rather than to interrupt the process of clonogenic cell division repeatedly (apoptosis), as in routine radiotherapy.

There are two forms of local radiotherapy, external beam radiotherapy and internal radiotherapy. External beam radiotherapy (EBRT) is the most common form of radiotherapy. An external source of ionizing radiation is pointed at a particular part of the body of the patient. In contrast to brachytherapy (sealed source radiotherapy) and unsealed source radiotherapy, in which the radiation source is inside the body, external beam radiotherapy directs the radiation at the tumor from outside the body. Orthovoltage (“superficial”) X-rays are used for treating skin cancer and superficial structures. X-rays and electron beams are by far the most widely used sources for external beam radiotherapy.

Internal radiotherapy (brachytherapy) is a form of radiotherapy where a sealed radiation source is placed inside or next to the area requiring treatment. The advantage of brachytherapy is that the irradiation affects only a very localized area around the radiation sources. Exposure to radiation of healthy tissues far away from the sources is therefore reduced and the tumor can be treated with very high doses of localised radiation whilst reducing the probability of unnecessary damage to surrounding healthy tissues. In addition, if the patient moves or if there is any movement of the tumor within the body during treatment, the radiation sources retain their correct position in relation to the tumor.

In a preferred embodiment the present invention provides the combination therapy as described herein, wherein the radiation therapy comprises local radiation therapy selected from external beam radiation or brachytherapy. In another preferred embodiment the present invention provides the combination therapy as described herein, wherein the radiation therapy comprises local hypofractionated radiation therapy. In yet another preferred embodiment the present invention provides the combination therapy as described herein, wherein the radiation therapy comprises local hypofractionated radiation at one or several doses in the range of 1 to 20 Gy, particularly in the range of 5 to 20 Gy. In yet another preferred embodiment the present invention provides the combination therapy as described herein, wherein the radiation therapy comprises one to three doses of local hypofractionated radiation in the range of 5 to 10 Gy. In yet another preferred embodiment the present invention provides the combination therapy as described herein, wherein the radiation therapy comprises local hypofractionated radiation at one dose in the range of 8 to 10 Gy, preferably at 8 or 10 Gy. In yet another preferred embodiment the present invention provides the combination therapy as described herein, wherein the radiation therapy comprises local hypofractionated radiation at three doses in the range of 5 to 10 Gy, preferably at 8 Gy.

The radiation therapy as defined herein can be applied in accordance with any schedule known to a person of skill in the art such as, for example, a clinical oncologist specialized in radiation therapy for cancer treatment. In one embodiment, the radiation therapy is administered after components a) and b).

In another embodiment, components a) and b) as defined herein are administered at day 1, and the radiation therapy is administered once at day 2 of a treatment cycle, for example a treatment cycle from 21 to 28 days, preferably 28 days.

In yet another embodiment, components a) and b) as defined herein are administered at day 1, and the radiation therapy is administered at day 2, followed by additional administrations every 5 to 7 days up to the end of a treatment cycle, for example, a treatment cycle from 21 to 28 days, preferably 28 days.

### Treg Depletion via CD25 Binding

As used herein “Treg cell depletion therapy” or “Treg depletion therapy” means a treatment regimen that results in the reduction of Tregs in the subject as compared to the level of Tregs in the subject before the therapy. Compounds that deplete Treg cells (i.e. “Treg cell depleting agents”) are known in the art. The depletion of Tregs can be measured by techniques known in the art for example as disclosed in WO2018/167104 and Simpson et al (2013) J Exp Med 210, 1695-710. In one embodiment the “Treg cell depleting agent” is an anti-CD25 antibody as defined herein.

As used herein, “regulatory T cells” (“Treg”, “Treg cells”, or “Tregs”) refer to a lineage of CD4+T lymphocytes specialized in controlling autoimmunity, allergy and infection. Typically, they regulate the activities of T cell populations, but they can also influence certain innate immune system cell types. Tregs are usually identified by the expression of the biomarkers CD4, CD25 and Foxp3. Naturally occurring Treg cells normally constitute about 5-10% of the peripheral CD4+ T lymphocytes. However, within a tumour microenvironment (i.e. tumour-infiltrating Treg cells), they can make up as much as 20-30% of the total CD4+ T lymphocyte population.

CD25 is the alpha chain of the IL-2 receptor, and is found on activated T cells, regulatory T cells, activated B cells, some NK T cells, some thymocytes, myeloid precursors and oligodendrocytes. CD25 associates with CD122 and CD132 to form a heterotrimeric complex that acts as the high-affinity receptor for IL-2. The consensus sequence of human CD25 is identified by Uniprot accession number P01589 (herein as SEQ ID NO 11).

As used herein an “anti-CD25 antibody” or an “an antibody that binds CD25” refers to an antibody that is capable of binding to the CD25 subunit of the IL-2 receptor. This subunit is also known as the alpha subunit of the IL-2 receptor. In one aspect, an anti-CD25 antibody is an antibody capable of specific binding to the CD25 subunit (antigen) of the IL-2 receptor.

“Specific binding”, “bind specifically”, and “specifically bind” are understood to mean that the antibody has a dissociation constant (Kd) for the antigen of interest of less than about 10-6 M, 10-7 M, 10-8 M, 10-9 M, 10-10 M, 10-11 M, 10-12 M or 10-13 M. In a preferred embodiment, the dissociation constant is less than 10-8 M, for instance in the range of 10-9 M, 10-10 M, 10-11 M, 10-12 M or 10-13 M.

An anti-CD25 antibody suitable for use in the invention are antibodies that are capable of depleting or reducing Treg cells.

As used herein, references to “depleted” or “depleting” (with respect to the depletion of regulatory T cells by an anti-CD25 antibody agent) it is meant that the number, ratio or percentage of Tregs is decreased relative to when the antibody is not administered. In particular embodiments of the invention as described herein, over about 5%, 10%, 20%, 30%, 40%, 50%, 60%, 70%, 80%, 90% or 99% of the regulatory T cells are depleted.

Anti-CD25 antibodies that can deplete Treg cells and are suitable for use in the invention include for example those described in WO2017/174331, WO2018/167104, WO2019/008386, WO2019/175215, WO2019/175216, WO2019/175217, WO2019/175220, WO2019/17522. WO2019/175223, WO2019/175224, WO2019/175226, the contents of which are incorporated herein by reference.

In a preferred embodiment of the invention, the anti-CD25 antibody binds FcγR with high affinity, preferably an activating receptor with high affinity. Preferably the antibody binds FcγRI and/or FcγRIIa and/or FcγRIIIa with high affinity. In a particular embodiment, the antibody binds to at least one activatory Fcγ receptor with a dissociation constant of less than about 10-6M, 10-7 M, 10-8M, 10-9M or 10-10M.

In some embodiments, the antibody is an IgG1 antibody, preferably a human IgG1 antibody, which is capable of binding to at least one Fc activating receptor. For example, the antibody may bind to one or more receptor selected from FcγRI, FcγRIIa, FcγRIIc, FcγRIIIa and FcγRIIIb. In some embodiments, the antibody is capable of binding to FcγRIIIa. In some embodiments, the antibody is capable of binding to FcγRIIIa and FcγRIIa and optionally FcγRI. In some embodiments, the antibody is capable of binding to these receptors with high affinity, for example with a dissociation constant of less than about 10-7M, 10-8M, 10-9M or 10-10M.

In some embodiments, the antibody binds an inhibitory receptor, FcγRIIb, with low affinity. In some embodiments, the antibody binds FcγRIIb with a dissociation constant higher than about 10-7 M, higher than about 10-6 M or higher than about 10-5 M.

In some embodiments the anti-CD25 antibody may be afucosylated. The Fc region of the antibody can be modified to change the glycosylation profile using known techniques in the art.

Available techniques to produce antibodies with absent or reduced fucosylation profiles, include commercially available technologies such as GlyMAXX (ProBiogen) and methods such as those disclosed in WO2011/035884.

In some embodiments the anti-CD25 antibody induces ADCC activity. The anti-CD25 antibody exhibits ADCC activity against CD25+ target cells. “Antibody-dependent cell-mediated cytotoxicity” (ADCC) refers to a cell-mediated reaction in which nonspecific cytotoxic cells that express Fc receptors (FcRs) (e.g. Natural Killer (NK) cells, neutrophils, and macrophages) recognize bound antibody on a target cell and thereby lead to lysis of the target cell. In some embodiments the anti-CD25 antibody induces ADCP activity. “Antibody-dependent cell-mediated phagocytosis” (ADCP) refers to a cell-mediated reaction in which phagocytes (such as macrophages) that express Fc receptors (FcRs) recognize bound antibody on a target cell and thereby lead to phagocytosis of the target cell.

The anti-CD25 antibody used in the invention many function through ADCC and ADCP activity. ADCC and ADCP can be measured using assays that are known and available in the art.

In some embodiments of the invention the anti-CD25 antibody does not inhibit the binding of Interleukin-2 (IL-2) to CD25. References herein to “does not inhibit the binding of Interleukin-2 to CD25” may alternatively be expressed as the anti-CD25 antibody is a non-IL-2 blocking antibody or a “non-blocking” antibody (with respect to the non-blocking of IL-2 binding to CD25 in the presence of the anti-CD25 antibody), i.e. the antibody does not block the binding of Interleukin-2 to CD25 and in particular does not inhibit Interleukin-2 signalling in CD25-expressing cells. References herein to a non-IL-2 blocking antibody may alternatively be expressed as an anti-CD25 antibody that “does not inhibit the binding of Interleukin-2 to CD25” or as an anti-CD25 antibody that “does not inhibit the signalling of IL-2” or as anti-CD25NIB (NIB=Non-IL-2 Blocking). References to “non-blocking” “non-IL-2 blocking”, “does not block”, or “without blocking” and the like (with respect to the non-blocking of IL-2 binding to CD25 in the presence of the anti-CD25 antibody) include embodiments wherein the anti-CD25 antibody of the invention does not block the signalling of IL-2 via CD25. That is the anti-CD25 antibody inhibits less than 50% of IL-2 signalling compared to IL-2 signalling in the absence of the antibodies. In particular embodiments of the invention as described herein, the anti-CD25 antibody inhibits less than about 50%, 40%, 35%, 30%, preferably less than about 25% of IL-2 signalling compared to IL-2 signalling in the absence of the antibodies.

Some anti-CD25 antibodies may allow binding of IL-2 to CD25, but still block signalling via the CD25 receptor. The non-IL-2 blocking anti-CD25 antibodies allow binding of IL-2 to CD25 to facilitate at least 50% of the level of signalling via the CD25 receptor compared to the signalling in the absence of the anti-CD25 antibody.

IL-2 signalling via CD25 may be measured by methods as discussed for example in WO2018/167104 and as known in the art. Comparison of IL-2 signalling in the presence and absence of the anti-CD25 antibody agent can occur under the same or substantially the same conditions. In some embodiments, IL-2 signalling can be determined by measuring by the levels of phosphorylated STAT5 protein in cells, using a standard Stat-5 phosphorylation assay. For example, a Stat-5 phosphorylation assay to measure IL-2 signalling may involve culturing PMBC cells in the presence of the anti-CD25 antibody at a concentration of 10 ug/ml for 30 mins and then adding varying concentrations of IL-2 (for example 10 U/ml or vary concentrations of 0.25 U/ml, 0.74 U/ml, 2.22 U/ml, 6.66 U/ml or 20 U/ml) for 10 mins. Cells may then be permeabilized and levels of STAT5 protein can then be measured with a fluorescent labelled antibody to a phosphorylated STAT5 peptide analysed by flow cytometry. The percentage blocking of IL-2 signalling can be calculated as follows: % blocking=100×[(% Stat5+ cells No Antibody group−% Stat5+ cells 10 ug/ml Antibody group)/(% Stat5+ cells No Antibody group).

Examples of non-blocking anti-CD25 antibodies are described in WO2018/167104, WO2019/175215, WO2019/175216, WO2019/175217, WO2019/175220, WO2019/17522. WO2019/175223, WO2019/17524, WO2019/17526 the contents of which are incorporated herein by reference in their entirety.

The anti-CD25 antibody may specifically bind to an epitope within the extracellular region of human CD25. In some embodiments the antibody binds to an epitope that is distinct from the IL-2 binding site and and does not block the binding of IL-2 to CD25.

As used herein, “epitope” refers to a portion of an antigen that is bound by an antibody or antigen-binding fragment. As is well known in the art, epitopes can be formed both from contiguous amino acids (linear epitope) or non-contiguous amino acids juxtaposed by tertiary folding of a protein (conformational epitopes). Epitopes formed from contiguous amino acids are typically retained on exposure to denaturing solvents whereas epitopes formed by tertiary folding are typically lost on treatment with denaturing solvents. An epitope is conformational in that it is comprised of portions of an antigen that are not covalently contiguous in the antigen but that are near to one another in three-dimensional space when the antigen is in a relevant conformation. For example, for CD25, conformational epitopes are those comprised of amino acid residues that are not contiguous in CD25 extracellular domain; linear epitopes are those comprised of amino acid residues that are contiguous in CD25 extracellular domain. Means for determining the exact sequence and/or particularly amino acid residues of the epitope for the anti-CD25 antibody are known in the literature, including competition with peptides, from antigen sequences, binding to CD25 sequences from different species, truncated, and/or mutagenized (e.g. by alanine scanning or other site-directed mutagenesis), phage display-based screening, yeast presentation technologies, or (co-) crystallography techniques. Methods of determining spatial conformation of epitopes are also well known in the art and include, for example, x-ray crystallography and 2-D nuclear magnetic resonance. See, for example, Epitope Mapping Protocols in Methods in Molecular Biology, Vol. 66, Glenn E. Morris, Ed (1996). Therefore, in some embodiments the anti-CD25 antibody may recognise a conformational epitope.

In one embodiment, anti-CD25 antibodies in accordance with the present invention are disclosed in WO2019/175216, WO2019/175217 and WO2019/1175222.

In another embodiment, the anti-CD25 antibody in accordance with the present invention is the antibody disclosed as “aCD25-a-686” or “αCD25 Mab GlyMAXX” in WO2019/1175222. This antibody may sometimes also be referred to as RG6292.

In still another embodiment, the anti-CD25 antibody in accordance with the present invention is an afucosylated human IgG1 monoclonal antibody with a heavy chain (HC) sequence having the sequence of SEQ ID NO: 12 and a light chain (LC) sequence having the sequence of SEQ ID NO: 13. Such antibody is known to be “non-IL-2 blocking” i.e. it does not inhibit the binding of IL-2 to CD25 (see e.g. WO2019/1175222).

Variants of the above defined antibodies can also be used. Variants of the antibodies also include antibodies wherein the sequence for each of the light chain and heavy chains comprise an amino acid sequence with at least 80% identity thereto. The term “Percent (%) identity” as known in the art is the relationship between two or more polypeptide sequences or two or more polynucleotide sequences, as determined by comparing the sequences. In the art, identity also means the degree of sequence relatedness between polypeptide or polynucleotide sequences, as the case may be, as determined by the match between strings of such sequences. While there exist a number of methods to measure identity between two polypeptides or two polynucleotide sequences, methods commonly employed to determine identity are codified in computer programs. Preferred computer programs to determine identity between two sequences include, but are not limited to, GCG program package (Devereux, et al., Nucleic Acids Research, 12, 387 (1984), BLASTP, BLASTN, and FASTA (Atschul et al., J. Molec. Biol. 215, 403 (1990)). The percent identity of two amino acid sequences or of two nucleic acid sequences is determined by aligning the sequences for optimal comparison purposes (e.g., gaps can be introduced in the first sequence for best alignment with the sequence) and comparing the amino acid residues or nucleotides at corresponding positions. The “best alignment” is an alignment of two sequences which results in the highest percent identity. The percent identity is determined by the number of identical amino acid residues or nucleotides in the sequences being compared (i.e., % identity=number of identical positions/total number of positions×100). Generally, references to % identity herein refer to % identity along the entire length of the molecule, unless the context specifies or implies otherwise.

As used herein, the term “antibody” refers to both intact immunoglobulin molecules as well as fragments thereof that include the antigen-binding site, and includes polyclonal, monoclonal, genetically engineered and otherwise modified forms of antibodies, including but not limited to chimeric antibodies, humanised antibodies, heteroconjugate and/or multispecific antibodies (e.g., bispecific antibodies, diabodies, tribodies, and tetrabodies), and antigen binding fragments of antibodies, including e.g. Fab′, F(ab′)2, Fab, Fv, rlgG, polypeptide-Fc fusions, single chain variants (scFv fragments, VHHs, Trans-bodies®, Affibodies®, shark single domain antibodies, single chain or Tandem diabodies (TandAb®), VHHs, Anticalins®, Nanobodies®, minibodies, BiTE®s, bicyclic peptides and other alternative immunoglobulin protein scaffolds). In some embodiments, an antibody may lack a covalent modification (e.g., attachment of a glycan) that it would have if produced naturally. In some embodiments, an antibody may contain a covalent modification (e.g., attachment of a glycan, a detectable moiety, a therapeutic moiety, a catalytic moiety, or other chemical group providing improved stability or administration of the antibody, such as poly-ethylene glycol). In some embodiments, the antibody may be in the form of a masked antibody (e.g. Probodies®). A masked antibody can comprise a blocking or “mask” peptide that specifically binds to the antigen binding surface of the antibody and interferes with the antibody's antigen binding. The mask peptide is linked to the antibody by a cleavable linker (e.g. by a protease). Selective cleavage of the linker in the desired environment, i.e. in the tumour environment, allows the masking/blocking peptide to dissociate, enabling antigen binding to occur in the tumour, and thereby limiting potential toxicity issues. “Antibody” may also refer to camelid antibodies (heavy-chain only antibodies) and antibody-like molecules such as anticalins (Skerra (2008) FEBS J 275, 2677-83). In some embodiments, an antibody is polyclonal or oligoclonal, that is generated as a panel of antibodies, each associated to a single antibody sequence and binding more or less distinct epitopes within an antigen (such as different epitopes within human CD25 extracellular domain that are associated to different reference anti-human CD25 antibodies. Polyclonal or oligoclonal antibodies can be provided in a single preparation for medical uses as described in the literature (Kearns JD et al., 2015. Mol Cancer Ther. 14:1625-36).

The antibodies used in the present invention may be monospecific, bispecific, or multispecific. “Multispecific antibodies” may be specific for different epitopes of one target antigen or polypeptide, or may contain antigen-binding domains specific for more than one target antigen or polypeptide. In some embodiments of the invention the antibody is monospecific. In some embodiments the antibody binds CD25 in a monovalent manner. In some embodiments the antibody is a TCB as further defined herein.

In some embodiments of the invention the antibody is monoclonal. The antibody may additionally or alternatively be humanised or human. In a further embodiment, the antibody is human, or in any case an antibody that has a format and features allowing its use and administration in human subjects.

As used herein, “monoclonal antibody” is not limited to antibodies produced through hybridoma technology. The term “monoclonal antibody” refers to an antibody that is derived from a single clone, including any eukaryotic, prokaryotic, or phage clone, and not the method by which it is produced.

As used herein, “human antibody” refers to antibodies having variable regions in which both the framework and CDR regions are derived from human germline immunoglobulin sequences.

Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo).

Antibodies (Abs) and immunoglobulins (Igs) are glycoproteins having the same structural characteristics. Immunoglobulins may be from any class such as IgA, IgD, IgG, IgE or IgM. Immunoglobulins can be of any subclass such as IgG1, IgG2, IgG3, or IgG4. In a preferred embodiment of the invention the anti-CD25 antibody is from the IgG class, preferably the IgG1 subclass. In one embodiment, the anti-CD25 antibody is from the human IgG1 subclass.

The combination treatment in accordance with the present invention has valuable pharmaceutical properties, especially in the treatment of cancer. The term “cancer” can be a haematological cancer or a solid cancer. In some embodiments the cancer involves a solid tumor (e.g. a solid tumor cancer) and the treatment is for treating cancer or preventing the relapse of a solid tumour. In another embodiment, the treatment in accordance with the present invention is for reducing metastatic spread. As used herein, “solid tumors” are an abnormal growth or mass of tissue that usually does not contain cysts or liquid areas, in particular, tumors and/or metastasis (wherever located) other than leukaemia or non-solid lymphatic cancers. Solid tumors may be benign or malignant. Different types of solid tumors are named for the type of cells that form them and/or the tissue or organ in which they are located. Examples of solid tumors are sarcomas (including cancers arising from transformed cells of mesenchymal origin in tissues such as cancellous bone, cartilage, fat, muscle, vascular, hematopoietic, or fibrous connective tissues), carcinomas (including tumors arising from epithelial cells), mesothelioma, neuroblastoma, retinoblastoma, etc. In one embodiment a solid cancer (or tumor) is selected from breast cancer, lung cancer, colon cancer, ovarian cancer, melanoma cancer, bladder cancer, renal cancer, kidney cancer, liver cancer, head and neck cancer, colorectal cancer, pancreatic cancer, gastric carcinoma cancer, esophageal cancer, mesothelioma or prostate cancer. In some embodiments the cancer is selected from acute myeloid leukaemia, diffuse large cell B-Cell lymphoma, multiple myeloma, melanoma, non-small cell lung cancer, renal cancer, ovarian cancer, bladder cancer, pancreatic cancer, sarcoma and/or colorectal cancer.

In yet another embodiment, there is provided the combination for use as defined herein, wherein the solid tumor is selected from head and neck squamous cell carcinomas (HNSCC), pancreatic cancer, pancreatic ductal adenocarcinoma (PDAC), melanoma, lung cancer, kidney cancer, breast cancer, colon cancer, ovarian cancer, cervical cancer, liver cancer, prostate cancer, bladder cancer, gastric cancer, glioblastoma and sarcomas, preferably from head and neck squamous cell carcinomas (HNSCC), pancreatic cancer and pancreatic ductal adenocarcinoma (PDAC).

Reference to “treatment”, “treat” or “treating” a cancer as used herein defines the achievement of at least one positive therapeutic effect, such as for example, reduced number of cancer cells, reduced tumour size, reduced rate of cancer cell infiltration into peripheral organs, or reduced rate of tumour metastasis or tumour growth. Positive therapeutic effects in cancer can be measured in a number of ways (e.g. Weber (2009) J Nucl Med 50, 1S-10S). By way of example, with respect to tumour growth inhibition, according to National Cancer Institute (NCI) standards, a T/C % ratio of 42% is the minimum level of anti-tumour activity. A T/C<10% is considered a high anti-tumour activity level, with T/C (%)=Median tumour volume of the treated/Median tumour volume of the control×100.

References to “combination” or “in combination” herein refer to separate, simultaneous or sequential administration, unless the context specifies otherwise.

A “therapeutically effective amount” (or effective amount) as used herein is the amount of the respective compound or combination that will elicit the biological or medical response of a tissue, system, animal or human that is being sought by the researcher, veterinarian, medical doctor or other clinician. In some embodiments, a therapeutically effective amount is one that reduces the incidence and/or severity of, stabilizes, and/or delays onset of, one or more symptoms of the disease, disorder, and/or condition in accordance with the treatment regimen. Those of ordinary skill in the art will appreciate that a “therapeutically effective amount” does not in fact require successful treatment be achieved in a particular subject. In some embodiments, the treatment achieved by a therapeutically effective amount is any of progression free survival (PFS), disease free survival (DFS) or overall survival (OS). PFS, also referred to as “Time to Tumour Progression” indicates the length of time during and after treatment that the cancer does not grow, and includes the amount of time patients have experienced a complete response or a partial response, as well as the amount of time patients have experienced stable disease. DFS refers to the length of time during and after treatment that the patient remains free of disease. OS refers to a prolongation in life expectancy as compared to naive or untreated individuals or patients.

It is one aspect of the present invention to provide a combination therapy which enables, improves or enhances “effective treatment” of a patient, having cancer. In accordance with the present invention one condition of such “effective treatment” is the depletion of Treg cells in said patient. Thus, in one embodiment, there is provided the combination for use as defined herein, wherein the combination is used to reduce the overall tumor burden. In another embodiment, there is provided the combination for use as defined herein, wherein the combination is used to treat acquired resistance to a previous therapy against that same cancer in the same patient. In yet another embodiment, there is provided the combination for use as defined herein, wherein the combination is used to prevent formation of metastases, preferably lung metastases, or to decrease the metastatic spread.

In another embodiment, the present invention discloses a pharmaceutical product comprising the combination for use as defined herein together with instructions how to apply it.

A set of clauses defining the invention and its preferred aspects and embodiments is as follows:
1. A combination for use in the treatment of cancer, wherein the combination comprises a) a first component comprising an effective amount of a Treg cell depleting agent; and b) a second component comprising an effective amount of a bispecific immunocytokine; and c) a third component comprising an effective amount of radiation therapy, and wherein the components a) to c) are for simultaneous or sequential administration.
2. The combination for use according to clause 1, wherein the Treg cell depleting agent is an anti-CD25 antibody.
3. The combination for use according to clause 2, wherein the anti-CD25 antibody is a monoclonal antibody which does not inhibit the binding of IL2 to CD25.
4. The combination for use according to anyone of clauses 1 to 3, wherein the bispecific immunecytokine is an antibody which blocks PD-1/PD-L1 signaling axis while simultaneously binding IL2 receptors on the same cell (PD1-IL2v).
5. The combination for use according to clause 4, wherein the IL2 binding domain (IL2v) is a mutated variant specific to CD122.
6. The combination for use according to any one of clauses 1 to 5, wherein the radiation therapy comprises local radiation therapy selected from external beam radiation or brachytherapy.
7. The combination for use according to any one of clauses 1 to 6, wherein the radiation therapy comprises local hypofractionated radiation therapy.
8. The combination for use according to any one of clauses 1 to 7, wherein the radiation therapy comprises local hypofractionated radiation at one or several doses in the range of 1 to 20 Gy, particularly in the range of 5 to 20 Gy.
9. The combination for use according to clause 8, wherein the radiation therapy comprises one to three doses of local hypofractionated radiation in the range of 5 to 10 Gy.
10. The combination for use according to clause 8 or 9, wherein the radiation therapy comprises local hypofractionated radiation at one dose in the range of 8 to 10 Gy, preferably at 8 or 10 Gy.
11. The combination for use according to clause 8 or 9, wherein the radiation therapy comprises local hypofractionated radiation at three doses in the range of 5 to 10 Gy, preferably at 8 Gy.
12. The combination for use according to any one of clauses 1 to11, wherein the radiation therapy is administered after components a) and b).
13. The combination for use according to clause 12, wherein components a) and b) are administered at day 1, and the radiation therapy is administered once at day 2 of a treatment cycle.
14. The combination for use according to clause 12, wherein components a) and b) are administered at day 1, and the radiation therapy is administered at day 2, followed by additional administrations every 5 to 7 days up to the end of a treatment cycle.
15. The combination for use according to any one of clauses 1 to 14, wherein the cancer is a solid tumor.
16. The combination for use according to clause 15, wherein the solid tumor is selected from Head and neck squamous cell carcinomas (HNSCC), pancreatic cancer, pancreatic ductal adenocarcinoma (PDAC), melanoma, lung cancer, kidney cancer, breast cancer, colon cancer, ovarian cancer, cervical cancer, liver cancer, prostate cancer, bladder cancer, gastric cancer, glioblastoma and sarcomas.
17. The combination for use according to clause 15, wherein the solid tumor is selected from Head and neck squamous cell carcinomas (HNSCC), pancreatic cancer and pancreatic ductal adenocarcinoma (PDAC).
18. The combination for use according to any one of clauses 1 to 17, wherein the combination is used to reduce the overall tumor burden.
19. The combination for use according to any one of clauses 1 to 17, wherein the combination is used to treat acquired resistance to a previous therapy against that same cancer in the same patient.
20. The combination for use according to any one of clauses 1 to 17, wherein the combination is used to prevent formation of metastases, preferably lung metastases, or to decrease the metastatic spread.
21. A pharmaceutical product comprising the combination for use according to any one of clauses 1 to 20 together with instructions how to apply it.
22. A method for treating a patient with cancer, comprising administering an effective amount of a combination according to any one of clauses 1 to 20.
23. The combination for use according to any one of clauses 1 to 20, the pharmaceutical product according to clause 21, or the method according to clause 22 wherein component a) is optional.
24. The combination for use according to any one of clauses 1 to 20, the pharmaceutical product according to clause 21, or the method according to clause 22 which does not comprise component a).

### EXAMPLES

### Materials and Methods

### Study Design

The objective of the study was to determine how PD1-IL2v, anti-CD25, and radio therapy (RT) modulate the anti-tumor immune response, and the subsequent effects on (i) primary and (ii) distance tumor development. These objectives relied upon initial utilization of publicly available ssRNA-seq data sets (n=44), RNA-seq and CyTOF analysis from a recently completed phase 1 trial (n=16) to provide justification for immunological targets. Main techniques used were flow cytometry (n=5-6), histology (n=4), proteomics/metabolomics (n=3-5), ELISA (n=6/sample). Mice for in vivo studies were age and gender matched and randomly assigned to treatment groups. Samples sizes were determined to achieve statistical significance and endpoints were determined by IACUC protocols.

### Cell Lines

MOC2, LY2, and P029 murine squamous cell carcinoma cells lines were used for the in vivo studies. Cell lines were cultured in appropriate media; DMEM-F12 with 10% FBS and 1% primocin/fungin for LY2 and P029, and a 1:2 mixture of DMEM-F12/IMDM supplemented with 10% FBS and 1% primocin/fungin, 1.75 ug EGF, 20 ug hydrocortisone, and 0.1% insulin for MOC2, as previously reported.

P029 cell line was provided in collaboration with XJ Wang at University of Colorado Anschutz Medical Campus, Department of Pathology and was generated as follows:

Establishing P029 cell line: K15-CrePR1, LSL-Kras^{G12D} and Smad4^{f/f} mice on a C57BL/6J background were interbred and tail snips genotyped to establish tri-genic mice with Cre recombinase driven by the keratin15 promoter which activates expression of oncogenic mutant Kras^{G12D} and deletes the Smad4 tumor suppressor in stratified epithelia as previously described⁷⁰ Female mouse P029 developed a spontaneous skin lesion on the cervical region that was allowed to reach a diameter of 2 cm at which time the mouse was sacrificed and tumor harvested for histological evaluation and cell line generation. The tumor was minced with scalpels, dissociated on a gentleMACS Tissue Dissociator using C tubes (Miltenyi) and incubated 40 min at 37° C. in 1 mg/mL Type II Collagenase (Worthington). Tissue suspensions were rinsed in PBS using centrifugation between washes and initially cultured in complete media (DMEM/F12 media containing 10% FBS and 1×primocin antibiotics) for 7 days. To encourage epithelial cell growth and reduce fibroblast growth, cells were cultured in serum free keratinocyte media (Gibco) supplemented with 2 ng/mL EGF and 1×primocin. A stable, proliferating cell line, P029, was established after 2 weeks of culture and 4 passages to expand epithelial cells and eliminate fibroblasts. To verify tumor establishment and metastasis capabilities, P029 cell line was transplanted to the flanks of recipient female C57BL/6J mice using 50,000 cells in 50% matrigel/50% PBS (Corning) and monitored for 6 weeks when tumors reached 2 cm in diameter. Full necropsy and histological evaluation demonstrated P029 cells metastasize to the lung, liver, and lymph node.

Murine KPC pancreatic cancer cell lines PK5L1940 and FC1242 were passaged in RPMI1640 supplemented with 10% FBS. Cells were passaged every 2-3 days at a density of 1:4-1:10. Cells were not allowed to grow beyond passage 20.

### Mouse Models and Tumor Studies

C57B1/6 and Balb/c mice were obtained from the Jackson Laboratory (Bar Harbor, Maine, USA) and Charles River (Wilmington, MA, USA) respectively and were used for MOC2, P029, and LY2 in vivo studies. Mice were housed with a maximum of 5 per cage and all protocols for animal models were approved by the Institutional Animal Care and Use Committee (IACUC) of the University of Colorado Anschutz Medical Campus. Murine MOC2, LY2, and P029 were implanted orthotopically into the buccal mucosa as previously described⁷. Mice were appropriately age matched and were randomized into groups, with treatment beginning when tumor volume was approximately 150 mm³. Tumor measurements were conducted twice weekly using digital calipers, with tumor volume was calculated as V=(A×B²)/2. A is measured as the short diameter and B as the long diameter. For tumor studies the following cell numbers were implanted into the buccal mucosa: MOC2 cell line 1×10⁵ cells were implanted per mouse, LY2 1×10⁶ cells were implanted per mouse, and for P029 5×10⁴ cells were implanted per mouse. Primary tumor, serum, and lungs were harvested at the time of sacrifice.

Female C57BL6 (6 weeks old) were purchased from Jackson Laboratories (Indianapolis, In, USA). All mice were cared for in accordance with the ethical guidelines and conditions set and overseen by the University of Colorado, Anschutz Medical Campus Animal Care and Use Committee. Protocols used for animal studies were reviewed and approved by the Institutional Animal Care and Use committee at the University of Colorado, Anschutz Medical Campus.

Local orthotopic implantations were conducted by first anesthetizing mice using isoflurane and making a 1 cm incision in the left subcostal region. Mouse pancreata were located, externalized, and injected with 200,000 PK5L1940 or FC1242 KPC cells suspended 1:1 in Matrigel (Corning, Corning, NY). Pancreata were then reintroduced into abdomen and mice peritoneum and skin were closed. Protocol described in further detail (Qiu and Su, 2013). Survival and flow cytometric in vivo studies were conducted and analyzed separately.

Metastatic orthotopic implantations were conducted as above with spleen externalization following subcostal incision. Spleens were first ligated with horizon clips and 1 hemispleen was injected with 200,000 KPC cells suspended in 50 μl 10% RPMI followed by washout injection of 50 μl PBS. Pancreatic vessels were then ligated with horizon clips and hemispleen was excised prior to closure of peritoneum and skin. Metastatic implantation described in further detail (Soares et al., 2014). For cancer specific mortality, mice determined to have died from other causes were excluded from the analysis.

Tumor rechallenging was conducted by injecting either PK5L1940 cells at a final concentration of 5×10⁵ cells/0.1 ml or FC1242 cells at a final concentration of 1×10⁶ cells/0.1 ml into the right flank of each animal. Flank tumors were measured twice weekly with digital calipers and tumor volumes were estimated using the formula (V=A×B²/2 mm³), where A is the longer and B is the shorter diameter of the tumor.

Prior to adoptive transfer experiments, CD8 T cells were sorted with a CD8-negative selection kit (StemCellTechnologies cat#19853) per manufacturer guidance. Cells were counted, resuspended into 100uL PBS, and injected via tail vein into restrained mice.

### Antibodies and Drugs

aCD25 (anti-CD25), PD1-IL2v, and DP47-IL2v were provided in collaboration with Roche Pharmaceuticals. aCD25 is RG6292 as defined herein or a mouse surrogate antibody of RG6292. PD1-IL2v or its mouse surrogate is used as defined herein (see e.g. SEQ ID Nos 5-10). DP47-IL2v is a non-targeted version of the IL2v moiety, used as control. aCD25 was given at a concentration of 3 mg/kg, PD1-IL2v and DP47-IL2v were given at a concentration of 0.5 mg/kg. Antibodies were administered weekly via I.P. injection beginning one day prior to the beginning of RT. For studies not utilizing radiation therapy, I.P. injections were administered after tumor implantation, at a time point equivalent to one day before RT. aPD-1 (Clone: 29F.1A12) and αNK1.1 (Clone: PK136) were administered twice weekly at 10 mg/kg via I.P. injections. NK cell depletion was verified using flow cytometric analysis. All dilutions were made using sterile DPBS (Gibco).

### Irradiation

Irradiation was performed using the PXi-225Cx image guided irradiator at 225 kV, 20 mA with a 0.3 mm Cu filter. Mice were anaesthetized with vaporized isoflurane and placed in the prone position, and RT was delivered at a dose rate of 5.6 Gy/min. Dose rates are checked monthly using an ionization chamber and CBCT scans were acquired to determine accurate positioning of mice. Irradiation plan was based on Monte Carlo simulations of a mouse model.

Specifically for the PDAC models (Examples 3 and 4): Image-guided radiotherapy was performed using the X-Rad SmART small animal irradiator (Precision X-Ray, North Bradford CT) at 225 k Vp, 20 mA with 0.3 mm Cu filter. Mice were positioned in the prone orientation and a CT scan was acquired. Radiation was delivered at a dose rate of 5.6 Gy/min. A single 8 Gy dose of X-ray radiation was delivered to mouse pancreata using 10 mm square beam with field edges at mouse midline and below left ribs. Monte-Carlo simulation was performed using SmART-ATP software (SmART Scientific Solutions, Maastricht, Netherlands) with a CBCT scan of one mouse to determine the appropriate time and current. All mice received identical treatment after repositioning by fluoroscopy. For all in vivo experiments, radiation was given at 7 days post-implantation.

### Flow Cytometry

Tumor, blood, and tumor draining lymph nodes were harvested and processed for flow cytometric analysis. Tumor tissue was chopped and incubated in Collagenase III (Worthington) for 30 mins at 37° C. After incubation, tissue was passed through a 70 um nylon cell strainer to produce a single cell suspension. After centrifugation, red blood cells were lysed using RBC lysis buffer (Invitrogen), using HBSS to neutralize the lysis buffer. Lymph nodes were similarly processed by mechanical separation into single cell suspension. Blood was immediately centrifuged after collection and resuspended in RBC lysis buffer as described above. Cells were transferred into 24 well plates and incubated with monensis and brefeldin to prevent release of cytokines, and stimulated with PMA/ionomycin cocktail for 4 hours at 37° C. Following incubation, cells were incubated in FC block (CD16/CD32 antibody, Tonbo bioscience) for 15 mins at 4° C. Cells were then incubated in Live/Dead Fixable Aqua Viability Stain Kit (Invitrogen) in the dark for 20 mins at 4° C. Cells were then stained for surface markers and incubated for 20 mins at 4° C. For analysis of immune cells, the following antibodies were: PerCP-CD45 (clone: 30-F11, Biolegend), BUV805-CD3 (clone: 17A2, BD Biosciences), BUV 496-CD4 (clone: GK1.5, BD Biosciences), BB515-CD8 (clone: 53-6.7, BS Biosciences), BV570-CD44 (clone: IM7, Biolegend), Pe-Cy7-NKp46 (clone: 29A1.4, Biolegend), Superbright 436-CD69 (clone: H1.2F3, eBioscience), BV605-DNAM-1 (clone: TX42.1, Biolegend), BV786-CD25 (clone: 3C7, BD Biosciences), BUV395-PD-1 (clone: J43, BD Biosciences), BV650-PD-L1 (clone: MIH5, BD BioSciences), PE-Cy5-CD11c (clone: N418, Biolegend), PE/Dazzle 594-MHC II (clone M5/114.15.2, Biolegend), PerCP-Cy5.5-CD80 (clone: 16-10A1, Biolegend), BUV661-CD11b (clone: M1/70, BD Biosciences), Alexa Fluor 647-Ly6C (clone: HK1.4, Biolegend), BV421-Ly6G (clone: 1A8, Biolegend), Alexa Fluor 700-CD19 (clone: 6D5, Biolegend), BV480-F4/80 (clone: T45-2342), eFluor 450-iNOS (clone: CXNFT, eBioscience), PE-CD163 (clone: S15049F, Biolegend), APC-eFluor 780-Ki-67 (clone: SolA15, eBioscience), Alexa Fluor 532-Foxp3 (clone: FjK-16s, eBioscience), APC-IL-2 (clone: JES6-5H4, eBioscience), BUV737-IFNγ (clone: XMG1.2, BD Biosciences), FITC-Granzyme B (clone: QA16A02, Biolegend), BV750-TNFα (clone: MP6-XT22, Biolegend), BV711-IL-10 (clone: JES5-16E3, BD Bioscience), PE-pan-cytokeratin (clone: AE-1/AE-3, Novus Biologicals), BV650-EpCAM (clone: G8.8, Biolegend), Alexafluor 488-pan cytokeratin (clone: AE1/AE3, Invitrogen) Alexafluor 700-NK1.1 (clone: S17016D, Biolegend), eFluor450-CD122 (clone: TM-b1, eBioscience), PE/Dazzle594-CD25 (clone: C37, Biolegend), Alexafluor 647-CD11b (clone: M1/70, Biolegend), BV711-NKG2D (clone: CX5, BD Bioscience), PerCP-eFluor 710-NKG2A (clone: 20d5, eBioscience), BUV661-Ly49H (clone: 3D10, BD Bioscience), PerCP-Cy5.5-KLRG1 (clone: 2F1, BD Bioscience), BV750-OX40 (clone: OX-40, BD Bioscience), PE-LFA-1 (clone: H155-78, Biolegend), Superbright 436-CXCR3 (clone: CXCR3-173, eBioscience), APC-CD137 (clone: 17B5, eBioscience), BV421-NKG2I (clone: 854929, BD Bioscience), FITC-Ly49G2 (clone: 4D11, eBioscience), BV786-Ly49A (clone: A1, BD Bioscience). After surface staining, cells were fixed and permeabilized using the Foxp3 perm/fix kit (Invitrogen) overnight. Following incubation, cells were stained for intracellular markers and incubated for 30 mins at 4° C. Samples were then run on a Cytek Aurora spectral cytometer at the University of Colorado Diabetes Research Center Flow Cytometry Core. Fluorescence minus one controls were used to determine gating strategy. Flowjo analysis software was used for data analysis.

### Immunohistochemistry and H&E Staining

Lungs were harvested at time of sacrifice and embedded in OCT medium and frozen at −80° C. to preserve tissue. Frozen tissue was submitted to University of Colorado Anschutz Medical Campus, Gates Center for Regenerative Medicine Histology Core where slides were cut, mounted, and H&E stained. Tissue was mounted using every 3^{rd} cut.

### NK Cytotoxicity Assay

NK cells were harvested and isolated from the blood of tumor bearing C57BL/6 mice using an NK negative selection isolation kit (Stemcell). P029 tumor cells were stained with calcein (Thermofisher) at a concentration of 2 ug/mL in RPMI media with 10% FBS. NK cells and P029 tumor cells were incubated together at a 2:1 ratio of NK to tumor cells and incubated at 37° C. for 4 hours. After incubation plates were centrifuged and supernatant was removed and transferred to 96 well plate. Plates were read on Tecan Infinite M plex fluorescence plate reader at 485 nm excitation and 530 nm emission. Cell lysis was calculated using the following equation: [(Test release-spontaneous release)/(Maximum release-spontaneous release)]×100. Maximum release was calculated by incubating P029 tumor cells with solution of 1% Triton×100 in RPMI and spontaneous release was calculated by incubating cells in incubation media without stimulus added.

### Imagestream Cytometry

NK cells were harvested and isolated from spleens of tumor bearing mice. Processed into single cell suspension following above protocol, and NK were isolated using an NK isolation kit (Stemcell) following manufacturer's instructions. Isolated NK's were then stained using the following antibodies: APC-EpCAM, PE/Dazzle 594-NKp46 (Biolegend), FITC-Granzyme B (Biolegend). Nuclei were stained using 4′6-diamidino-2-phenylindole (DAPI). Following staining, cells were washed and resuspended in PBS and processed through Amnis Imagestream X Mk II imaging flow cytometer (Amnis, Seattle, WA). Analysis was performed using IDEAS 6.2 software (AMNIS, Seattle, WA).

### Metabolomics and Proteomics

Blood from tumor bearing mice was collected and processed into a single cell suspension as described above. After processing, CD8 and NK cells were isolated using a CD8 T cell (Stemcell) and NK cell (Militenyi) negative isolation kits following manufacturer's instructions. Isolated cells were then stained with PE-CD8 and APC-NKp46 and then cell sorted to achieve a pure single cell population. Cell sorting was performed using a Beckman Coulter MoFlo XDP750 (Beckman Coutler). High-throughput metabolomics analysis was performed at the University of Colorado School of Medicine Metabolomics Facility on murine serum samples. Samples were thawed on ice and metabolites extracted from serum by adding chilled 5:3:2 methanol: acetonitrile: water (v/v/v) to each tube, 1:24 serum: buffer (v/v), followed subsequently by 30 minutes of vortexing and 10 minutes of centrifugation both at 4° C. as described⁷². All supernatants were analyzed twice (10 uL injections each) by ultra-high-performance liquid chromatography using a Thermo Vanquish UHPLC coupled to a Thermo Q Exactive mass spectrometer in negative and positive polarity modes. For each method, the UHPLC utilized a Phenomenex C18 column at a flow rate of 0.45 mL/min for 5 minutes. Samples entered the MS by electrospray ionization; full technical details are described previously⁷³. Data was analyzed using Maven (1.4.20-dev-772) and quality controls were maintained as described^{72,74}. Data was normalized by sum and auto scaled to generate PCA, PLS-DA, and heatmaps with MetaboAnalyst (5.0).

The samples were digested according to the FASP protocol using a 10 kDa molecular weight cutoff filter. In brief, the samples were mixed in the filter unit with 8 M urea, 0.1M ammonium bicarbonate (AB) pH 8.0, and centrifuged at 14 000 g for 15 min. The proteins were reduced with 10 mM DTT for 30 min at RT, centrifuged, and alkylated with 55 mM iodoacetamide for 30 min at RT in the dark. Following centrifugation, samples were washed 3× with urea solution, and 3× with 50 mM AB, pH 8.0. Protein digestion was carried out with sequencing grade modified Trypsin (Promega) at 1/50 protease/protein (wt/wt) at 37° C. overnight. Peptides were recovered from the filter using 50 mM AB. A 20 ul of each sample was loaded onto individual Evotips for desalting and then washed with 20 μL 0.1% FA followed by the addition of 100 μL storage solvent (0.1% FA) to keep the Evotips wet until analysis. The Evosep One system ((Evosep, Odense, Denmark) was used to separate peptides on a Pepsep column, (150 um inter diameter, 15 cm) packed with ReproSil C18 1.9 um, 120A resin. The system was coupled to the timsTOF Pro mass spectrometer (Bruker Daltonics, Bremen, Germany) via the nano-electrospray ion source (Captive Spray, Bruker Daltonics). The mass spectrometer was operated in PASEF mode. The ramp time was set to 100 ms and 10 PASEF MS/MS scans per topN acquisition cycle were acquired. MS and MS/MS spectra were recorded from m/z 100 to 1700. The ion mobility was scanned from 0.7 to 1.50 Vs/cm². Low-abundance precursor ions with an intensity above a threshold of 500 counts but below a target value of 20000 counts were repeatedly scheduled and otherwise dynamically excluded for 0.4 min. The identification settings were as follows: Trypsin, Specific, with a maximum of 2 missed cleavages, up to 2 isotope errors in precursor selection allowed for, 10.0 ppm as MS1 and 0.4 Da as MS2 tolerances; fixed modifications: Carbamidomethylation of C (+57.021464 Da), variable modifications: Oxidation of M (+15.994915 Da), Acetylation of protein N-term (+42.010565 Da), Pyrolidone from peptide N-term Q or C (−17.026549 Da). Graphs were rendered using Graphpad Prism (9.3.1).

### Statistical Analysis

All statistical analyses were processed using GraphPad Prism v9. Statistical analysis was completed using one-way analysis of variance (one-way ANOVA) with Tukey correction for comparisons with 3 or more groups, and unpaired t-tests for comparisons using only two groups. Kaplan-Meier curves were used for survival analysis, using log-ranked (Mantel-Cox) test for comparisons of all groups.

### Example 1: Treatment with αCD25 and PD1-IL2v Results in Tumor Growth Delay in HNSCC Tumors

This example tests the hypothesis that targeting both PD-1 and IL-2 signaling concurrently would increase treatment efficacy. The murine immunocytokine PD1-IL2v (muPD1-IL2v) which blocks PD-1/PD-L1 signaling axis while simultaneously binding IL-2 receptors on the same cell was used. The mutated IL-2v binding domain is unique in that it is specific to CD122, allowing it to bypass CD25 mediated Treg activation³¹⁻³³. However, Tregs can also express CD122, and past work has demonstrated that depletion of Tregs is vital to overcoming radioresistance and treatment response. Considering this, a novel αCD25 antibody³⁴, optimized to interact with activating Fc regions to facilitate enhanced depletion of intratumoral Tregs, while leaving IL-2 signaling intact was used. This approach allows for greater depletion of tumor infiltrating Tregs, while selectively expanding and activating CD25 deficient cells, namely CD8 T cells and NKs³⁴.

The combination of RT and αCD25 eradicated LY2 tumors in approximately 80% of mice (FIG. 1A, B). Interestingly, in the Treg dependent LY2 model muPD1-IL2v with or without radiotherapy does not harbor significant therapeutic benefit on local tumor progression, further demonstrating the necessity of Treg depletion in this model. In the MOC2 tumor model, a significant reduction in tumor growth was observed with the addition of muPD1-IL2v to RT, with further improved survival upon the addition of aCD25 (FIG. 1C, D). These treatments were further tested in the newly developed Kras/smad4/p53 mutated metastatic P029 tumor model. All combinations of RT with αCD25 and muPD1-IL2v exhibited tumor growth delay (FIG. 1E), and all combinations showed increased survival compared to RT alone (FIG. 1F).

### Example 2: Reduction of Lung Metastasis

Given the increased systemic activation of circulating lymphocytes and NK cells, this example investigates the functional implication of this increase on systemic tumor spread. The P029 model was used due to its high predilection for widespread metastasis, especially to the lungs. The examined lung tissue was collected from orthotopically implanted P029 tumors, which were harvested at time of sacrifice (FIG. 2A). A decrease in the percentage of mice displaying gross metastatic lung lesions was observed in the groups treated with muPD1-IL2v (FIG. 2B). Cancer cells which break off from the primary tumor enter circulation and can seed tumors at distant sites²⁴. Analysis of circulating tumor cells (CTCs), showed that the percentage of CD45 cells expressing EpCAM or cytokeratin, both markers of circulating tumor cells, were significantly reduced in the groups receiving muPD1-IL2v in contrast to RT alone and RT+αCD25 (FIG. 2C, D). These data suggest that muPD1-IL2v assists in preventing metastatic spread and formation of distant lesions by decreasing the amount of tumor cells in circulation, even in the context of local tumor progression.

Example 3: aPD1-IL2v+RT+aCD25 Results in Superior Response in a PDAC Mouse Model

This example uses orthotopic mouse models of Kras driven PDAC with PK5L1940 and FC1242 KPC cell lines to examine the effect of the murine variant antibody fusion protein, aPD1-IL2v in combination with radio therapy and Treg depletion, on pancreatic cancer growth. Using the PK5L1940 cell line, the addition of aCD25 to RT and aPD1-IL2v antibody treatment marginally improved survival (FIG. 3A). Response to treatment was also driven by aPD1-IL2v using the KPC-derived, Kras-driven FC1242 cell line (FIG. 3B).

### Example 4: RT+aPD1-IL2v Combination Treatment Results in Activation of Peripheral CTLs, Reduced Metastatic Burden in PDAC Models

This example investigates the metastatic burden following RT plus aPD1-IL2v treatment by flow cytometric analysis on circulating immune populations. Due to their role in controlling metastatic spread^{22, 38}, peripheral NK cells were analyzed. Although the frequency of circulating NK cells was decreased following aCD25 and aPD1-IL2v treatment, expression of the functional markers DNAM1 and Gnzmb were increased with the addition of aPD1-IL2v (FIG. 4A), suggesting aPD1-IL2v may also function to reduce metastatic burden by activating NK cells in the periphery.

To directly test the effect of this immune modulation on metastasis and disease dissemination, this example next utilized a hemi-splenectomy metastatic model of pancreatic cancer wherein tumor cells are injected through splenic vessels and reproducibly form metastatic lesions in the liver⁷⁶. The data from using this model with the PK5L1940 cell line are shown in FIG. 4B. Furthermore, a complete response was observed in ⅛ mice in the RT+aPD1-IL2v treatment group and 2/8 mice in the RT+aCD25+aPD1-IL2v treatment group, highlighting the robust and systemic nature of the aPD1-IL2v induced anti-tumor immune response. Complete response with these therapy combinations was also achieved using the KPC FC1242 cell line in the hemi-splenectomy metastatic model (FIG. 4C)

### References

1. Sambi M, Bagheri L, Szewczuk MR. Current challenges in cancer immunotherapy: Multimodal approaches to improve efficacy and patient response rates. J Oncol. 2019;2019.
doi: 10.1155/2019/4508794
2. Nör JE, Gutkind JS. Head and Neck Cancer in the New Era of Precision Medicine. J Dent Res. 2018;97(6):601-602. doi:10.1177/0022034518772278
3. Mandal R, Şenbabaoğlu Y, Desrichard A, et al. The head and neck cancer immune landscape and its immunotherapeutic implications. JCI Insight. 2016;1(17). doi:10.1172/jci.insight.89829
4. Karam SD, Raben D. Radioimmunotherapy for the treatment of head and neck cancer. Lancet Oncol. 2019;20(8):e404-e416. doi:10.1016/S1470-2045(19)30306-7
5. Waldman AD, Fritz JM, Lenardo MJ. A guide to cancer immunotherapy: from T cell basic science to clinical practice. Nat Rev Immunol. 2020;20(11):651-668. doi:10.1038/s41577-020-0306-5
6. Botticelli A, Mezi S, Pomati G, et al. The 5-Ws of immunotherapy in head and neck cancer. Crit Rev Oncol Hematol. 2020;153:103041. doi:10.1016/J.CRITREVONC.2020.103041
7. Oweida A, Hararah MK, Phan A, et al. Resistance to radiotherapy and PD-L1 blockade is mediated by TIM-3 upregulation and regulatory T-cell infiltration. Clin Cancer Res. 2018;24(21):5368-5380.
doi:10.1158/1078-0432.CCR-18-1038
8. Oweida AJ, Darragh L, Phan A, et al. STAT3 Modulation of Regulatory T Cells in Response to Radiation Therapy in Head and Neck Cancer. J Natl Cancer Inst. 2019;111(12):1339-1349. doi:10.1093/jnci/djz036
9. Ihara F, Sakurai D, Horinaka A, et al. CD45RA-Foxp3high regulatory T cells have a negative impact on the clinical outcome of head and neck squamous cell carcinoma. Cancer Immunol Immunother. 2017;66(10):1275-1285. doi:10.1007/S00262-017-2021-Z
10. Bickett TE, Knitz M, Darragh LB, et al. FLT3L Release by Natural Killer Cells Enhances Response to Radioimmunotherapy in Preclinical Models of HNSCC. Clin Cancer Res. 2021;27(22):6235-6249. doi: 10.1158/1078-0432.ccr-21-0971
11. Martin JF, Perry JSA, Jakhete NR, Wang X, Bielekova B. An IL-2 Paradox: Blocking CD25 on T Cells Induces IL-2-Driven Activation of CD56 bright NK Cells. J Immunol. 2010;185 (2):1311-1320.
doi:10.4049/jimmunol.0902238
12. Navarro AG, Björklund AT, Chekenya M. Therapeutic potential and challenges of natural killer cells in treatment of solid tumors. Front Immunol. 2015;6(APR). doi:10.3389/fimmu.2015.00202
13. Vivier E, Tomasello E, Baratin M, Walzer T, Ugolini S. Functions of natural killer cells. Nat Immunol. 2008;9(5):503-510. doi:10.1038/ni1582
14. Sim GC, Radvanyi L. The IL-2 cytokine family in cancer immunotherapy. Cytokine Growth Factor Rev. 2014;25(4):377-390. doi:10.1016/J.CYTOGFR.2014.07.018
15. Wu Y, Tian Z, Wei H. Developmental and functional control of natural killer cells by cytokines. Front Immunol. 2017;8(AUG). doi:10.3389/fimmu.2017.00930
16. Koyama S, Akbay EA, Li YY, et al. Adaptive resistance to therapeutic PD-1 blockade is associated with upregulation of alternative immune checkpoints. Nat Commun. 2016;7.
doi:10.1038/ncomms10501
17. Sharpe AH, Pauken KE. The diverse functions of the PD1 inhibitory pathway. Nat Rev Immunol. 2018;18(3):153-167. doi:10.1038/nri.2017.108
18. Kumagai S, Togashi Y, Kamada T, et al. The PD-1 expression balance between effector and regulatory T cells predicts the clinical efficacy of PD-1 blockade therapies. doi:10.1038/s41590-020-0769-3
19. Kamada T, Togashi Y, Tay C, et al. PD-1+ regulatory T cells amplified by PD-1 blockade promote hyperprogression of cancer. Proc Natl Acad Sci U S A. 2019;116(20):9999-10008.
doi:10.1073/PNAS.1822001116/-/DCSUPPLEMENTAL
20. Umaña P, Deak LC, Ahmed R, et al. Differentiating PD-1+stem-like CD8 T cells towards distinct effectors with enhanced therapeutic potential by an engineered IL-2 cis-targeted to PD-1.
doi:10.21203/rs.3.rs-329812/v1
21. Chinen T, Kannan AK, Levine AG, et al. An essential role for the IL-2 receptor in T reg cell function. Nat Immunol. 2016;17(11):1322-1333. doi: 10.1038/ni.3540
22. López-Soto A, Gonzalez S, Smyth MJ, Galluzzi L. Control of Metastasis by NK Cells. Cancer Cell. 2017;32(2):135-154. doi: 10.1016/J.CCELL.2017.06.009
23. Sharma P, Kumar P, Sharma R. Natural killer cells—Their role in tumour immunosurveillance. J Clin Diagnostic Res. 2017;11(8):BE01-BE05. doi:10.7860/JCDR/2017/26748.10469
24. Lo HC, Xu Z, Kim IS, et al. Resistance to natural killer cell immunosurveillance confers a selective advantage to polyclonal metastasis. Nat Cancer. 2020;1(7):709-722. doi:10.1038/s43018-020-0068-9
25. Puram S V., Tirosh I, Parikh AS, et al. Single-Cell Transcriptomic Analysis of Primary and Metastatic Tumor Ecosystems in Head and Neck Cancer. Cell. 2017;171(7):1611-1624.e24.
doi:10.1016/j.cell.2017.10.044
26. Cillo AR, Kürten CHL, Tabib T, et al. Immune Landscape of Viral-and Carcinogen-Driven Head and Neck Cancer. Immunity. 2020;52(1):183-199.e9. doi:10.1016/j.immuni.2019.11.014
27. Boyman O, Sprent J. The role of interleukin-2 during homeostasis and activation of the immune system. Nat Rev Immunol. 2012;12(3):180-190. doi:10.1038/nri3156
28. Zimmer J, Andrès E, Hentges F. NK cells and Treg cells: A fascinating dance cheek to cheek. Eur J Immunol. 2008;38(11):2942-2945. doi:10.1002/eji.200838813
29. Ralainirina N, lie Poli A, Michel T, et al. Control of NK cell functions by CD4 CD25 regulatory T cells. doi:10.1189/jlb.0606409
30. Wang X, Lupardus P, Laporte SL, Garcia KC. Structural Biology of Shared Cytokine Receptors. Published online 2009. doi:10.1146/annurev.immunol.24.021605.090616
31. Ben-Shmuel A, Biber G, Barda-Saad M. Unleashing Natural Killer Cells in the Tumor Microenvironment—The Next Generation of Immunotherapy? Front Immunol. 2020;11.
doi:10.3389/fimmu.2020.00275
32. Waldhauer I, Gonzalez-Nicolini V, Freimoser-Grundschober A, et al. Simlukafusp alfa (FAP-IL2v) immunocytokine is a versatile combination partner for cancer immunotherapy. MAbs. 2021;13(1).
doi:10.1080/19420862.2021.1913791
33. Klein C, Waldhauer I, Nicolini VG, et al. Cergutuzumab amunaleukin (CEA-IL2v), a CEA-targeted IL-2 variant-based immunocytokine for combination cancer immunotherapy: Overcoming limitations of aldesleukin and conventional IL-2-based immunocytokines. Oncoimmunology. 2017;6(3). doi:10.1080/2162402X.2016.1277306
34. Solomon I, Amann M, Goubier A, et al. CD25-Treg-depleting antibodies preserving IL-2 signaling on effector T cells enhance effector activation and antitumor immunity. Nat Cancer. 2020;1(12):1153-1166. doi:10.1038/s43018-020-00133-0
35. Knitz MW, Bickett TE, Darragh LB, et al. Targeting resistance to radiation-immunotherapy in cold HNSCCs by modulating the Treg-dendritic cell axis. J Immunother Cancer. 2021;9(4).
doi:10.1136/jitc-2020-001955
36. Overacre-Delgoffe AE, Chikina M, Dadey RE, et al. Interferon-γ Drives Treg Fragility to Promote Anti-tumor Immunity. Cell. 2017;169(6):1130-1141.e11. doi:10.1016/J.CELL.2017.05.005
37. Mempel TR, Henrickson SE, Von Andrian UH. T-Cell Priming by Dendritic Cells in Lymph Nodes Occurs in Three Distinct Phases.; 2004. www.nature.com/nature
38. Nakamura K, Smyth MJ. Immunoediting of cancer metastasis by NK cells. Nat Cancer. 2020;1(7):670-671. doi:10.1038/s43018-020-0081-z
39. Cong J. Metabolism of Natural Killer Cells and Other Innate Lymphoid Cells. Front Immunol. 2020;11.doi:10.3389/fimmu.2020.01989
40. O'Brien KL, Finlay DK. Immunometabolism and natural killer cell responses. Nat Rev Immunol. 2019;19(5):282-290. doi:10.1038/s41577-019-0139-2
41. Choi C, Finlay DK. Optimising NK cell metabolism to increase the efficacy of cancer immunotherapy. Stem Cell Res Ther. 2021;12(1). doi:10.1186/s13287-021-02377-8
42. Waldhauer I, Steinle A. NK cells and cancer immunosurveillance. Oncogene. 2008;27(45):5932-5943. doi:10.1038/onc.2008.267
43. Paul S, Lal G. The molecular mechanism of natural killer cells function and its importance in cancer immunotherapy. Front Immunol. 2017;8(SEP). doi:10.3389/fimmu.2017.01124
44. Fiegler N, Textor S, Arnold A, et al. Downregulation of the activating NKp30 ligand B7-H6 by HDAC inhibitors impairs tumor cell recognition by NK cells. Published online 2013.
doi:10.1182/blood-2013-02
45. López-Soto A, Huergo-Zapico L, Acebes-Huerta A, Villa-Alvarez M, Gonzalez S. NKG2D signaling in cancer immunosurveillance. Int J Cancer. 2015;136(8):1741-1750.
doi:10.1002/ijc.28775
46. Duan S, Guo W, Xu Z, et al. Natural killer group 2D receptor and its ligands in cancer immune escape. Mol Cancer. 2019;18(1). doi:10.1186/s12943-019-0956-8
47. Somanchi SS, McCulley KJ, Somanchi A, Chan LL, Lee DA. A novel method for assessment of natural killer cell cytotoxicity using image cytometry. PLoS One. 2015;10(10).
doi:10.1371/journal.pone.0141074
48. Nishimura H, Nose M, Hiai H, Minato N, Honjo T. Development of Lupus-like Autoimmune Diseases by Disruption of the PD-1 Gene Encoding an ITIM Motif-Carrying Immunoreceptor. Immunity. 1999;11(2):141-151. doi:10.1016/S1074-7613 (00) 80089-8
49. Simon S, Labarriere N. PD-1 expression on tumor-specific T cells: Friend or foe for immunotherapy? Oncoimmunology. 2018;7(1). doi:10.1080/2162402X.2017.1364828
50. Miller AM, Lundberg K, Özenci V, et al. CD4+CD25 high T Cells Are Enriched in the Tumor and Peripheral Blood of Prostate Cancer Patients. J Immunol. 2006;177(10):7398-7405.
doi:10.4049/jimmunol.177.10.7398
51. TAKENAKA M, SEKI N, TOH U, et al. FOXP3 expression in tumor cells and tumor-infiltrating lymphocytes is associated with breast cancer prognosis. Mol Clin Oncol. 2013;1(4):625-632.
doi:10.3892/mco.2013.107
52. O'callaghan DS, Rexhepaj E, Gately K, et al. Tumour islet Foxp3+T-cell infiltration predicts poor outcome in nonsmall cell lung cancer. Eur Respir J. 2015;46:1762-1772.
doi:10.1183/09031936.00159515
53. Sun D-S, Miao-Qing Zhao., Xia M, Li., Jiang L. Y-H. The correlation between tumor-infiltrating Foxp3+ regulatory T cells and cyclooxygenase-2 expression and their association with recurrence in resected head and neck cancers. doi:10.1007/s12032-011-9903-2
54. Arce Vargas F, Furness AJS, Solomon I, et al. Fc-Optimized Anti-CD25 Depletes Tumor-Infiltrating Regulatory T Cells and Synergizes with PD-1 Blockade to Eradicate Established Tumors. Immunity. 2017;46(4):577-586. doi:10.1016/J.IMMUNI.2017.03.013
55. Pisani P, Airoldi M, Allais A, et al. Metastatic disease in head & neck oncology. Acta Otorhinolaryngol Ital. 2020;40(2):S1-S86. doi:10.14639/0392-100X-suppl.1-40-2020
56. Beckham TH, Leeman JE, Xie P, et al. Long-term survival in patients with metastatic head and neck squamous cell carcinoma treated with metastasis-directed therapy. Br J Cancer. 2019;121(11):897-903. doi:10.1038/s41416-019-0601-8
57. Merzoug L Ben, Marie S, Satoh-Takayama N, et al. Conditional ablation of NKp46+ cells using a novel Ncr1greenCre mouse strain: NK cells are essential for protection against pulmonary B16 metastases. Eur J Immunol. 2014;44(11):3380-3391. doi:10.1002/eji.201444643
58. Glasner A, Ghadially H, Gur C, et al. Recognition and Prevention of Tumor Metastasis by the NK Receptor NKp46/NCR1. J Immunol. 2012;188(6):2509-2515. doi:10.4049/jimmunol.1102461
59. Glasner A, Isaacson B, Viukov S, et al. Increased NK cell immunity in a transgenic mouse model of NKp46 overexpression. Sci Rep. 2017;7(1). doi:10.1038/s41598-017-12998-w
60. Guerra N, Tan YX, Joncker NT, et al. NKG2D-Deficient Mice Are Defective in Tumor Surveillance in Models of Spontaneous Malignancy. Immunity. 2008;28(4):571-580.
doi:10.1016/J.IMMUNI.2008.02.016
61. Pasero C, Gravis G, Guerin M, et al. Inherent and tumor-driven immune tolerance in the prostate microenvironment impairs natural killer cell antitumor activity. Cancer Res. 2016;76(8):2153-2165. doi:10.1158/0008-5472.CAN-15-1965
62. Pearce EL, Pearce EJ. Metabolic pathways in immune cell activation and quiescence. Immunity. 2013;38(4):633-643. doi:10.1016/j.immuni.2013.04.005
63. Wang Z, Guan D, Wang S, Chai LYA, Xu S, Lam KP. Glycolysis and Oxidative Phosphorylation Play Critical Roles in Natural Killer Cell Receptor-Mediated Natural Killer Cell Functions. Front Immunol. 2020;11.doi:10.3389/fimmu.2020.00202
64. Gardiner CM. NK cell metabolism. doi:10.1002/JLB.MR0718-260R
65. Sheppard S, Santosa EK, Lau CM, et al. Lactate dehydrogenase A-dependent aerobic glycolysis promotes natural killer cell anti-viral and anti-tumor function. Cell Rep. 2021;35 (9).
doi:10.1016/j.celrep.2021.109210
66. Sun Y, Xu Z, Jiang J, Xu T, Xu J, Liu P. High Expression of Succinate Dehydrogenase Subunit A Which Is Regulated by Histone Acetylation, Acts as a Good Prognostic Factor of Multiple Myeloma Patients. Front Oncol. 2020;10. doi:10.3389/fonc.2020.563666
67. Satoh-Takayama N, Vosshenrich CAJ, Lesjean-Pottier S, et al. Microbial Flora Drives Interleukin 22 Production in Intestinal NKp46+ Cells that Provide Innate Mucosal Immune Defense. Immunity. 2008;29(6):958-970. doi:10.1016/J.IMMUNI.2008.11.001
68. Verrier T, Satoh-Takayama N, Serafini N, Marie S, Di Santo JP, Vosshenrich CAJ. Phenotypic and Functional Plasticity of Murine Intestinal NKp46 + Group 3 Innate Lymphoid Cells. J Immunol. 2016;196(11):4731-4738. doi:10.4049/jimmunol.1502673
69. Hudspeth K, Silva-Santos B, Mavilio D. Natural cytotoxicity receptors: Broader expression patterns and functions in innate and adaptive immune cells. Front Immunol. 2013;4 (MAR).
doi:10.3389/fimmu.2013.00069
70. White RA, Neiman JM, Reddi A, et al. Epithelial stem cell mutations that promote squamous cell carcinoma metastasis. J Clin Invest. 2013;123.doi:10.1172/JCI65856
71. Sun D, Wang J, Han Y, et al. TISCH: A comprehensive web resource enabling interactive single-cell transcriptome visualization of tumor microenvironment. Nucleic Acids Res. 2021;49(D1):D1420-D1430. doi:10.1093/nar/gkaa1020
72. Nemkov T, Hansen KC, D'Alessandro A. A three-minute method for high-throughput quantitative metabolomics and quantitative tracing experiments of central carbon and nitrogen pathways. Rapid Commun Mass Spectrom. 2017;31(8):663-673. doi:10.1002/rcm.7834
73. Nemkov T, Reisz JA, Gehrke S, Hansen KC, D'Alessandro A. High-Throughput Metabolomics: Isocratic and Gradient Mass Spectrometry-Based Methods. In: ; 2019:13-26. doi:10.1007/978-1-4939-9236-2_2
74. Gehrke S, Rice S, Stefanoni D, et al. Red Blood Cell Metabolic Responses to Torpor and Arousal in the Hibernator Arctic Ground Squirrel. Published online 2019.
doi:10.1021/acs.jproteome.9b00018

75. Akhmedov M, Martinelli A, Geiger R, Kwee I. Omics Playground: a comprehensive self-service la0tform for visualization, analytics and exploration of Big Omics Data. NAR Genomics Bioinforma. 2020;2(1). doi:10.1093/nargab/lqz019
76. Soares, K.C., Foley, K., Olino, K., Leubner, A., Mayo, S.C., Jain, A., Jaffee, E., Schulick, R.D., Yoshimura, K., Edil, B., and Zheng, L. (2014). A preclinical murine model of hepatic metastases. J Vis Exp, 51677. 10.3791/51677.

Sequences

| Description | Sequence | SEQ ID NO |
|---|---|---|
| Heavy chain | EVQLLESGGGLVQPGGSLRLSCAASGFSFSSYTMS | 1 |
| variable domain | WVRQAPGKGLEWVATISGGGRDIYYPDSVKGRFTI | |
| VH of anti-PD-1 | SRDNSKNTLYLQMNSLRAEDTAVYYCVLLTGRVY | |
| | FALDSWGQGTLVTVSS | |
| Light chain | DIVMTQSPDSLAVSLGERATINCKASESVDTSDNSF | 2 |
| variable domain | IHWYQQKPGQSPKLLIYRSSTLESGVPDRFSGSGSG | |
| VL of anti-PD-1 | TDFTLTISSLQAEDVAVYYCQQNYDVPWTFGQGT | |
| | KVEIK | |
| quadruple | APASSSTKKTQLQLEHLLLDLQMILNGINNYKNPK | 3 |
| mutant human | LTRMLTAKFAMPKKATELKHLQCLEEELKPLEEVL | |
| IL2 (IL-2qm or | NGAQSKNFHLRPRDLISNINVIVLELKGSETTFMCE | |
| IL2v) | YADETATIVEFLNR WITFAQSIISTLT | |
| WT human IL-2 | APTSSSTKKTQLQLEHLLLDLQMILNGINNYKNPKL | 4 |
| | TRMLTFKFYMPKKATELKHLQCLEEELKPLEEVLN | |
| | LAQSKNFHLRPRDLISNINVIVLELKGSETTFMCEY | |
| | ADETATIVEFLNR WITFAQSIISTLT | |
| PD-1 IL2v-HC | EVQLLESGGGLVQPGGSLRLSCAASGFSFSSYTMS | 5 |
| with IL2v (Fc | WVRQAPGKGLEWVATISGGGRDIYYPDSVKGRFTI | |
| knob, | SRDNSKNTLYLQMNSLRAEDTAVYYCVLLTGRVY | |
| LALAPG) | FALDSWGQGTLVTVSSASTKGPSVFPLAPSSKSTSG | |
| | GTAALGCLVKDYFPEPVTVSWNSGALTSGVHTFPA | |
| | VLQSSGLYSLSSVVTVPSSSLGTQTYICNVNHKPSN | |
| | TKVDKKVEPKSCDKTHTCPPCPAPEAAGGPSVFLF | |
| | PPKPKDTLMISRTPEVTCVVVDVSHEDPEVKFNWY | |
| | VDGVEVHNAKTKPREEQYNSTYRVVSVLTVLHQD | |
| | WLNGKEYKCKVSNKALGAPIEKTISKAKGQPREPQ | |
| | VYTLPPCRDELTKNQVSLWCLVKGFYPSDIAVEWE | |
| | SNGQPENNYKTTPPVLDSDGSFFLYSKLTVDKSRW | |
| | QQGNVFSCSVMHEALHNHYTQKSLSLSPGGGGGS | |
| | GGGGSGGGGSAPASSSTKKTQLQLEHLLLDLQMIL | |
| | NGINNYKNPKLTRMLTAKFAMPKKATELKHLQCL | |
| | EEELKPLEEVLNGAQSKNFHLRPRDLISNINVIVLEL | |
| | KGSETTFMCEYADETATIVEFLNRWITFAQSIISTLT | |
| PD-1 IL2v-HC | EVQLLESGGGLVQPGGSLRLSCAASGFSFSSYTMS | 6 |
| without IL2v | WVRQAPGKGLEWVATISGGGRDIYYPDSVKGRFTI | |
| (Fc hole, | SRDNSKNTLYLQMNSLRAEDTAVYYCVLLTGRVY | |
| LALAPG) | FALDSWGQGTLVTVSSASTKGPSVFPLAPSSKSTSG | |
| | GTAALGCLVKDYFPEPVTVSWNSGALTSGVHTFPA | |
| | VLQSSGLYSLSSVVTVPSSSLGTQTYICNVNHKPSN | |
| | TKVDKKVEPKSCDKTHTCPPCPAPEAAGGPSVFLF | |
| | PPKPKDTLMISRTPEVTCVVVDVSHEDPEVKENWY | |
| | VDGVEVHNAKTKPREEQYNSTYRVVSVLTVLHQD | |
| | WLNGKEYKCKVSNKALGAPIEKTISKAKGQPREPQ | |
| | VCTLPPSRDELTKNQVSLSCAVKGFYPSDIAVEWES | |
| | NGQPENNYKTTPPVLDSDGSFFLVSKLTVDKSRWQ | |
| | QGNVFSCSVMHEALHNHYTQKSLSLSP | |
| PD-1 IL2v-LC | DIVMTQSPDSLAVSLGERATINCKASESVDTSDNSF | 7 |
| | IHWYQQKPGQSPKLLIYRSSTLESGVPDRESGSGSG | |
| | TDFTLTISSLQAEDVAVYYCQQNYDVPWTFGQGT | |
| | KVEIKRTVAAPSVFIFPPSDEQLKSGTASVVCLLNN | |
| | FYPREAKVQWKVDNALQSGNSQESVTEQDSKDST | |
| | YSLSSTLTLSKADYEKHKVYACEVTHQGLSSPVTK | |
| | SFNRGEC | |
| Murine | EVQLQESGPGLVKPSQSLSLTCSVTGYSITSSYRWN | 8 |
| surrogate PD-1 | WIRKFPGNRLEWMGYINSAGISNYNPSLKRRISITR | |
| IL2v-HC with | DTSKNQFFLQVNSVTTEDAATYYCARSDNMGTTPF | |
| IL2v | TYWGQGTLVTVSSAKTTPPSVYPLAPGSAAQTNSM | |
| (P1AA6923) | VTLGCLVKGYFPEPVTVTWNSGSLSSGVHTFPAVL | |
| | QSDLYTLSSSVTVPSSTWPSQTVTCNVAHPASSTKV | |
| | DKKIVPRDCGCKPCICTVPEVSSVFIFPPKPKDVLTI | |
| | TLTPKVTCVVVAISKDDPEVQFSWFVDDVEVHTAQ | |
| | TKPREEQINSTFRSVSELPIMHQDWLNGKEFKCRV | |
| | NSAAFGAPIEKTISKTKGRPKAPQVYTIPPPKEQMA | |
| | KDKVSLTCMITNFFPEDITVEWQWNGQPAENYDN | |
| | TQPIMDTDGSYFVYSDLNVQKSNWEAGNTFTCSVL | |
| | HEGLHNHHTEKSLSHSPGGGGGSGGGGSGGGGSA | |
| | PASSSTSSSTAEAQQQQQQQQQQQQHLEQLLMDL | |
| | QELLSRMENYRNLKLPRMLTAKFALPKQATELKD | |
| | LQCLEDELGPLRHVLDGTQSKSFQLEDAENFISNIR | |
| | VTVVKLKGSDNTFECQFDDESATVVDFLRRWIAFA | |
| | QSIISTSPQ | |
| Murine | EVQLQESGPGLVKPSQSLSLTCSVTGYSITSSYRWN | 9 |
| surrogate PD-1 | WIRKFPGNRLEWMGYINSAGISNYNPSLKRRISITR | |
| IL2v-HC | DTSKNQFFLQVNSVTTEDAATYYCARSDNMGTTPF | |
| without IL2v | TYWGQGTLVTVSSAKTTPPSVYPLAPGSAAQTNSM | |
| (P1AA6923) | VTLGCLVKGYFPEPVTVTWNSGSLSSGVHTFPAVL | |
| | QSDLYTLSSSVTVPSSTWPSQTVTCNVAHPASSTKV | |
| | DKKIVPRDCGCKPCICTVPEVSSVFIFPPKPKDVLTI | |
| | TLTPKVTCVVVAISKDDPEVQFSWFVDDVEVHTAQ | |
| | TKPREEQINSTFRSVSELPIMHQDWLNGKEFKCRV | |
| | NSAAFGAPIEKTISKTKGRPKAPQVYTIPPPKKQMA | |
| | KDKVSLTCMITNFFPEDITVEWQWNGQPAENYKN | |
| | TQPIMKTDGSYFVYSKLNVQKSNWEAGNTFTCSVL | |
| | HEGLHNHHTEKSLSHSPGK | |
| Murine | DIVMTQGTLPNPVPSGESVSITCRSSKSLLYSDGKT | 10 |
| surrogate PD-1 | YLNWYLQRPGQSPQLLIYWMSTRASGVSDRESGSG | |
| IL2v-LC | SGTDFTLKISGVEAEDVGIYYCQQGLEFPTFGGGTK | |
| (P1AA6923) | LELKRTDAAPTVSIFPPSSEQLTSGGASVVCFLNNF | |
| | YPKDINVKWKIDGSERQNGVLNSWTDQDSKDSTY | |
| | SMSSTLTLTKDEYERHNSYTCEATHKTSTSPIVKSF | |
| | NRNEC | |
| Human CD25 | MDSYLLMWGLLTFIMVPGCQAELCDDDPPEIPHAT | 11 |
| sequence | FKAMAYKEGTMLNCECKRGFRRIKSGSLYMLCTG | |
| | NSSHSSWDNQCQCTSSATRNTTKQVTPQPEEQKER | |
| | KTTEMQSPMQPVDQASLPGHCREPPPWENEATERI | |
| | YHFVVGQMVYYQCVQGYRALHRGPAESVCKMTH | |
| | GKTRWTQPQLICTGEMETSQFPGEEKPQASPEGRPE | |
| | SETSCLVTTTDFQIQTEMAATMETSIFTTEYQVAVA | |
| | GCVFLLISVLLLSGLTWQRRQRKSRRTI | |
| HC sequence of | QVQLVQSGAEVKKPGSSVKVSCKASGGTFSSLAISWVR | 12 |
| RG6292 | QAPGQGLEWMGGIIPIFGTANYAQKFQGRVTITADESTS | |
| | TAYMELSSLRSEDTAVYYCARGGSVSGTLVDFDIWGQG | |
| | TMVTVSSASTKGPSVFPLAPSSKSTSGGTAALGCLVKDY | |
| | FPEPVTVSWNSGALTSGVHTFPAVLQSSGLYSLSSVVTV | |
| | PSSSLGTQTYICNVNHKPSNTKVDKKVEPKSCDKTHTCP | |
| | PCPAPELLGGPSVFLFPPKPKDTLMISRTPEVTCVVVDVS | |
| | HEDPEVKFNWYVDGVEVHNAKTKPREEQYNSTYRVVS | |
| | VLTVLHQDWLNGKEYKCKVSNKALPAPIEKTISKAKGQ | |
| | PREPQVYTLPPSRDELTKNQVSLTCLVKGFYPSDIAVEW | |
| | ESNGQPENNYKTTPPVLDSDGSFFLYSKLTVDKSRWQQ | |
| | GNVFSCSVMHEALHNHYTQKSLSLSPGK | |
| LC sequence of | DIQMTQSPSTLSASVGDRVTITCRASQSISSWLAWYQQK | 13 |
| RG6292 | PGKAPKLLIYKASSLESGVPSRFSGSGSGTEFTLTISSLQP | |
| | DDFATYYCQQYNIYPITFGGGTKVEIKRTVAAPSVFIFPP | |
| | SDEQLKSGTASVVCLLNNFYPREAKVQWKVDNALQSG | |
| | NSQESVTEQDSKDSTYSLSSTLTLSKADYEKHKVYACE | |
| | VTHQGLSSPVTKSFNRGEC | |

1. A combination for use in the treatment of cancer, wherein the combination comprises a) a first component comprising an effective amount of a Treg cell depleting agent; and b) a second component comprising an effective amount of a bispecific immunocytokine; and c) a third component comprising an effective amount of radiation therapy, and wherein the components a) to c) are for simultaneous or sequential administration.

2. The combination for use according to claim 1, wherein the Treg cell depleting agent is an anti-CD25 antibody.

3. The combination for use according to claim 2, wherein the anti-CD25 antibody is a monoclonal antibody which does not inhibit the binding of IL2 to CD25.

4. The combination for use according to anyone of claims 1 to 3, wherein the bispecific immunecytokine is an antibody which blocks PD-1/PD-L1 signaling axis while simultaneously binding IL2 receptors on the same cell (PD1-IL2v).

5. The combination for use according to claim 4, wherein the IL2 binding domain (IL2v) is a mutated variant specific to CD122.

6. The combination for use according to any one of claims 1 to 5, wherein the radiation therapy comprises local radiation therapy selected from external beam radiation or brachytherapy.

7. The combination for use according to any one of claims 1 to 6, wherein the radiation therapy comprises local hypofractionated radiation therapy.

8. The combination for use according to any one of claims 1 to 7, wherein the radiation therapy comprises local hypofractionated radiation at one or several doses in the range of 1 to 20 Gy, particularly in the range of 5 to 20 Gy.

9. The combination for use according to claim 8, wherein the radiation therapy comprises one to three doses of local hypofractionated radiation in the range of 5 to 10 Gy.

10. The combination for use according to claim 8 or 9, wherein the radiation therapy comprises local hypofractionated radiation at one dose in the range of 8 to 10 Gy, preferably at 8 or 10 Gy.

11. The combination for use according to claim 8 or 9, wherein the radiation therapy comprises local hypofractionated radiation at three doses in the range of 5 to 10 Gy, preferably at 8 Gy.

12. The combination for use according to any one of claims 1 to 11, wherein the radiation therapy is administered after components a) and b).

13. The combination for use according to claim 12, wherein components a) and b) are administered at day 1, and the radiation therapy is administered once at day 2 of a treatment cycle.

14. The combination for use according to claim 12, wherein components a) and b) are administered at day 1, and the radiation therapy is administered at day 2, followed by additional administrations every 5 to 7 days up to the end of a treatment cycle.

15. The combination for use according to any one of claims 1 to 14, wherein the cancer is a solid tumor.

16. The combination for use according to claim 15, wherein the solid tumor is selected from Head and neck squamous cell carcinomas (HNSCC), pancreatic cancer, pancreatic ductal adenocarcinoma (PDAC), melanoma, lung cancer, kidney cancer, breast cancer, colon cancer, ovarian cancer, cervical cancer, liver cancer, prostate cancer, bladder cancer, gastric cancer, glioblastoma and sarcomas.

17. The combination for use according to claim 15, wherein the solid tumor is selected from Head and neck squamous cell carcinomas (HNSCC), pancreatic cancer and pancreatic ductal adenocarcinoma (PDAC).

18. The combination for use according to any one of claims 1 to 17, wherein the combination is used to reduce the overall tumor burden.

19. The combination for use according to any one of claims 1 to 17, wherein the combination is used to treat acquired resistance to a previous therapy against that same cancer in the same patient.

20. The combination for use according to any one of claims 1 to 17, wherein the combination is used to prevent formation of metastases, preferably lung metastases, or to decrease the metastatic spread.

21. A pharmaceutical product comprising the combination for use according to any one of claims 1 to 20 together with instructions how to apply it.

22. A method for treating a patient with cancer, comprising administering an effective amount of a combination according to any one of claims 1 to 20.

23. The combination for use according to any one of claims 1 to 20, the pharmaceutical product according to claim 21, or the method according to claim 22, wherein component a) is optional.

24. The combination for use according to any one of claims 1 to 20, the pharmaceutical product according to claim 21, or the method according to claim 22 which does not comprise component a).


SEQUENCE LISTING (13 sequences)

SEQ ID NO: 1 - protein, 120 aa, synthetic construct
  source 1..120: note = Heavy chain variable domain VH of anti-PD-1
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSS

SEQ ID NO: 2 - protein, 111 aa, synthetic construct
  source 1..111: note = Light chain variable domain VL of anti-PD-1
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIK

SEQ ID NO: 3 - protein, 133 aa, synthetic construct
  source 1..133: note = quadruple mutant human IL2 (IL-2qm or IL2v)
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 4 - protein, 133 aa, Homo sapiens
  source 1..133: note = WT human IL-2
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 5 - protein, 597 aa, synthetic construct
  source 1..597: note = PD-1 IL2v - HC with IL2v (Fc knob, LALAPG)
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 6 - protein, 448 aa, synthetic construct
  source 1..448: note = PD-1 IL2v - HC without IL2v (Fc hole, LALAPG)
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 7 - protein, 218 aa, synthetic construct
  source 1..218: note = PD-1 IL2v - LC
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 607 aa, synthetic construct
  source 1..607: note = Murine surrogate PD-1 IL2v - HC with IL2v (P1AA6923)
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPGGGGGSGGGGSGGGGSAPASSSTSSSTAEAQQQQQQQQQQQQHLEQLLMDLQELLSRMENYRNLKLPRMLTAKFALPKQATELKDLQCLEDELGPLRHVLDGTQSKSFQLEDAENFISNIRVTVVKLKGSDNTFECQFDDESATVVDFLRRWIAFAQSIISTSPQ

SEQ ID NO: 9 - protein, 444 aa, synthetic construct
  source 1..444: note = Murine surrogate PD-1 IL2v - HC without IL2v (P1AA6923)
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPGK

SEQ ID NO: 10 - protein, 218 aa, synthetic construct
  source 1..218: note = Murine surrogate PD-1 IL2v - LC (P1AA6923)
DIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKRTDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 11 - protein, 272 aa, Homo sapiens
  source 1..272: note = Human CD25 sequence
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 12 - protein, 452 aa, synthetic construct
  source 1..452: note = HC sequence of RG6292
QVQLVQSGAEVKKPGSSVKVSCKASGGTFSSLAISWVRQAPGQGLEWMGGIIPIFGTANYAQKFQGRVTITADESTSTAYMELSSLRSEDTAVYYCARGGSVSGTLVDFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 214 aa, synthetic construct
  source 1..214: note = LC sequence of RG6292
DIQMTQSPSTLSASVGDRVTITCRASQSISSWLAWYQQKPGKAPKLLIYKASSLESGVPSRFSGSGSGTEFTLTISSLQPDDFATYYCQQYNIYPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC